(19) United States
(12) Patent Application Publication
(10) Pub. No.: US 2020/0316134 A1
(43) Pub. Date: Oct. 8, 2020

(54) Biological Scaffold Comprising Therapeutic Cells

(71) Applicant: UNIVERSITY OF MIAMI, Miami, FL (US)

(72) Inventors: Camillo Ricordi, Miami, FL (US); Dora M. Berman-Weinberg, Miami, FL (US); Marta Garcia Contreras, Miami, FL (US); Diego Correa, Miami, FL (US); Alice Tomei, Miami, FL (US)

(21) Appl. No.: 16/496,742

(22) PCT Filed: Mar. 26, 2018
(86) PCT No.: PCT/US18/24346

§ 371 Date: Sep. 23, 2019

Related U.S. Application Data
(60) Provisional application No. 62/476,742, filed on Mar. 25, 2017.

(51) Int. Cl. A61K 35/39; A61K 38/28; A61L 27/38; C12N 5/071; A61K 35/44; A61K 35/17; A61K 9/00; A61P 3/10

(52) U.S. Cl. CPC A61K 35/39; A61K 38/28; A61L 27/3804; C12N 5/0676; C12N 2501/20; A61K 35/17; A61K 9/0024; A61P 3/10; C12N 2501/10; A61K 35/44

(57) ABSTRACT

Methods of implanting therapeutic cells in a subject and methods of preparing pancreatic islet cells for implantation into a subject, prior to implantation into a subject, are provided herein.

### CROSS-REFERENCE TO RELATED APPLICATIONS

This application claims priority to U.S. Provisional Application No. 62/476,742, filed on Mar. 25, 2017, which is incorporated by reference in its entirety.

### BACKGROUND

Intrahepatic islet transplantation has been the gold standard for clinical islet transplantation trials aimed at treating patients with type 1 diabetes (T1D) and hypoglycemia unawareness or with surgically induced diabetes (pancreatectomy) (1). It has resulted in normalization of hemoglobin A_{1c}, improved glycemic control, and elimination of severe hypoglycemic events, even in the absence of insulin independence (2). Progressive graft dysfunction has been observed in clinical trials years after intrahepatic islet transplantation, often requiring reintroduction of exogenous insulin. Long-term intrahepatic islet dysfunction has been also observed in preclinical models (3). Activation of an immediate blood-mediated inflammatory reaction (IBMIR) and hypoxia in the transplant microenvironment after intrahepatic islet embolization contributes to functional impairment and the loss of a significant portion of the transplanted islets (4-7). Furthermore, the hepatic first pass of orally administered drugs exposes intrahepatic islets to higher concentrations of diabetogenic immunosuppressive agents. Other potential challenging factors in this setting include accumulation of peri-insular fat (microsteatosis) in the liver parenchyma (8-13). Moreover, chronic exposure of intrahepatic islets to endotoxins and other proinflammatory agents absorbed through the gastrointestinal tract, in addition to the IBMIR, may trigger adaptive immune responses that are known to be associated with a higher incidence of acute and chronic rejection episodes, as well as possibly facilitate recurrence of autoimmunity in transplanted subjects with T1D (2).

The final objective of developing an extrahepatic site for islet transplantation is not only the ability to provide physiologic portal drainage of endocrine pancreas secretions but also the possibility of engineering the transplant microenvironment for the development of successful biologic replacement strategies that could avoid the need for chronic recipient immunosuppression (14,15). Ideal characteristics of such a site include sufficient space to accommodate relatively large tissue volumes (e.g., low-purity or encapsulated insulin-producing cell products), allow for minimally invasive transplant procedures, and enable noninvasive longitudinal monitoring and access for graft biopsy and/or retrieval, as well as physiologic venous drainage through the portal system (14,15). An additional advantage is the reportedly immunomodulatory effect of antigens delivered though the portal venous system (portal tolerance) that was associated with lower rejection rates in pancreas transplants with portal versus systemically drained organs (16).

In view of the foregoing, there remains a need in the art for improved methods of islet transplantation.

### SUMMARY

Provided herein are data demonstrating that methods of implanting therapeutic cells can advantageously lead to long term treatment of metabolic diseases, such as Type I diabetes. Such data confirm that long term restoration of euglycemia and independence from exogenous insulin therapy without episodes of hypoglycemia can be successfully achieved in a human subject through implementation of the methods described herein. Data provided herein also demonstrate that therapeutic cells prepared according to methods of the present disclosures lead to increased glucose-stimulated insulin secretion which further aid in providing a safe means for treatment of subjects for metabolic diseases.

The present disclosure provides methods of implanting therapeutic cells in a subject. The therapeutic cells, in exemplary embodiments, produce and secrete a therapeutic agent, e.g., insulin. In exemplary embodiments, the method of implanting therapeutic cells in a subject comprises combining the therapeutic cells with a source of a first member of a cell matrix pair to create a therapeutic cell mixture; applying the therapeutic cell mixture to a surface of an organ in the subject; applying a source of a second member of the cell matrix pair to the surface of the organ over the therapeutic cell mixture; and applying a source of the first member of the cell matrix pair to the surface of the organ over the therapeutic cell mixture, thereby forming a therapeutic cell scaffold.

The present disclosure also provides a method of preparing pancreatic islet cells for implantation into a subject. In exemplary aspects, the method comprises culturing the pancreatic islet cells with endothelial-derived exosomes or endothelial cells.

The present disclosure further provides a method of preparing pancreatic islet cells for implantation into a subject. The method in exemplary embodiments comprises culturing the pancreatic islet cells with the contents of endothelial-derived exosomes prior to implanting the pancreatic islet cells into the subject. Accordingly, the method in exemplary instances is a method of preparing pancreatic islet cells for implantation into a subject, comprising culturing the pancreatic islet cells with hepatocyte growth factor (HGF), thrombospondin-1 (TSP-1), alaminin, a collagen, insulin growth factor binding protein-1 (IGFBP-2), CD40, IGFBP-1, sTNFRII, CD40L, TNFα, cIAP-2, IGFBP-3, TNFβ, CytoC, IGFBP-4, TRAIL R1, TRAIL R2, TRAIL R3, bad, IGF-1 sR, TRAIL R4, HSP60, p27, Caspase 8, IGF-2, or a combination thereof, prior to implanting the pancreatic islet cells into the subject.

Compositions comprising pancreatic islet cells in a subject, wherein the pancreatic islet cells are produced by the methods disclosed herein are furthermore provided. Also, the present disclosure provides methods of implanting the pancreatic islet cells produced by the methods disclosed herein.

The present disclosure provides yet another method of implanting pancreatic islet cells in a subject, comprising: combining pancreatic islet cells with a supporting cellular composition and a source of a first member of a cell matrix pair to create a therapeutic cell mixture; applying the therapeutic cell mixture to a surface of an organ in the subject; and applying a source of a second member of the cell matrix pair to the surface of the organ over the therapeutic cell mixture thereby forming a therapeutic cell scaffold. In exemplary instances, the method further comprises applying a source of the first member of the cell matrix pair to the surface of the organ over the therapeutic cell mixture.

### BRIEF DESCRIPTION OF THE DRAWINGS

FIGS. 1A-1C demonstrate intraomental islet implantation within a biologic scaffold. A: Schematic diagram of the transplant procedure. B: Procedure in rat. C: Procedure in NHP. After midline laparotomy (b1), the omentum is gently exteriorized and opened (b2 and c1). The islet graft, resuspended in autologous plasma (c2), is gently distributed onto the omentum (b3 and c3). Recombinant human thrombin is added onto the islets on the omental surface to induce gel formation (c4), and then the omentum is folded to increase the contact of the graft to the vascularized omentum (b4 and c5). Nonresorbable stitches were placed on the far outer margins of the graft in the NHP (c5) for easier identification of the graft area at the time of graft removal.

FIGS. 2A-2C demonstrate scanning electronic micrograph of the biologic scaffold in vitro. A: Plasma/thrombin mix. Fibrin polymerizes forming an intricate three-dimensional network (bar=5 μm). B: Untreated human islet cell surface in culture medium (bar=50 μm). C: Human islets embedded within the biologic scaffold. The polymerized fibrin forms an orthomorphic matrix around the islet surface (bar=50 μm).

FIGS. 3A-3G demonstrate intraomental islets transplanted into biologic scaffolds restore normoglycemia in diabetic rats. A: Nonfasting blood glucose levels in diabetic rats (n=7; 173.4±91 g body wt) transplanted with 3,000 IEQ (17,338±881 IEQ/kg) onto the omentum showing prompt reversal of diabetes and hyperglycemia after removal of the omental graft (arrowhead) on POD 74 (n=1) or 240 (n=4). B: Glycemic profile during IVGTT performed in selected animals (n=3) 2 months after transplant compared with that of naïve animals (n=3). Values shown are mean±SD. Inset shows area under the curve (AUC) (mg×min×dL⁻¹) for each group. C: Glycemic profile during OGTT performed in transplanted animals (n=5) at 11 (●) and 26 (∘) weeks after transplantation. Inset shows AUC (mg×min×dL⁻¹) during the glucose challenge. D-G: Representative histopathologic pattern of intraomental islet grafts. Sections were obtained from an intraomental islet graft explanted on POD 76. D: Hematoxylin-eosin staining. E: Masson trichrome staining. F and G: Immunofluorescence microscopy of a section stained with anti-insulin (INS) (red fluorescence), anti-GCG antibody (green fluorescence) (F), anti-SMA (green fluorescence) (G), and nuclear dye DAPI (blue fluorescence). The box indicates the area of the graft shown at higher magnification on the left panel. wk, week.

FIGS. 4A-4D demonstrate comparable function of intrahepatic and intraomental islets transplanted into biologic scaffolds. Nonfasting blood glucose levels in diabetic rats receiving a clinically relevant syngeneic islet mass of 1,300 IEQ (˜8,200 IEQ/kg body wt) within an intraomental biologic scaffold (A) (●, n=7) or into the liver (via the portal vein) (B) (∘, n=5) with islets from the same batch isolation. The groups had an identical time course for reversal of diabetes, and removal of the intraomental biologic scaffold on day 80 posttransplant resulted in return to hyperglycemia (arrowhead in A). Glycemic profile during OGTT performed in all transplanted animals 5 (C) or 11 weeks (D) after transplantation. Inset shows AUC (mg×min×dL⁻¹) during the glucose challenge for each group (wks, weeks).

FIGS. 5A-5G demonstrate biomarkers detected in the serum of rat recipients of intraomental biologic scaffold and intrahepatic syngeneic islets. Aliquots of 1,300 IEQ from the same syngeneic donor rat islet batch were transplanted in parallel either within the intraomental biologic scaffold (omentum [∘]) or the intrahepatic site (liver [●]). Blood samples were collected from indwelling JVC for detection of biomarker levels in circulation. Data presented are mean±SEM (n=4-7 per time point). A and B: Metabolic markers assessed at 1 h posttransplant. A: Insulin in μg/mL (*P=0.018). B: C-peptide in μg/mL. Inflammation markers assessed 24 h posttransplant: MCP-1/CCL2 in μg/mL (C), IL-6 in μg/mL (D), leptin in μg/mL (*P=0.013) (E), haptoglobin in μg/mL (F), and α2-macroglobulin in μg/mL (**P<0.03) (G). Inflammatory biomarkers MCP-1/CCL2 (FIG. 5C) and IL-6 (FIG. 5D) showed comparable increases between experimental groups at 24 h, with undetectable values by 72 h posttransplant in both groups (not shown). Leptin levels were significantly higher at 24 h in the recipients of the intrahepatic compared with intraomental group (633±31 vs. 483±35 μg/mL, respectively; P=0.013) (FIG. 5E). Acute-phase protein haptoglobin levels were comparable in both groups (FIG. 5F), while α2-macroglobulin levels were significantly higher in intrahepatic islet recipients versus the intraomental group at 24 h (280±58 vs. 155±26 μg/mL; one-tail t test: P<0.03) (FIG. 5G).

FIGS. 6A-6B demonstrate intraomental transplantation of islets with high and low purity into diabetic rats. A: Nonfasting blood glucose levels in diabetic rats transplanted with clinically relevant mass of 2,000 IEQ syngeneic islets with >95% purity (n=3) (167.3±1.5 g body wt [11,853±109 IEQ/kg]) or with 30% purity (n=3) (170.3±10.5 g body wt [11,771±725 IEQ/kg]) onto the omentum. Removal of the omental graft >100 days after transplantation (arrowhead) resulted in return to hyperglycemia. B: Glycemic profile during oral glucose tolerance test performed in animals transplanted with high-purity and low-purity islet preparations 70 days after transplantation.

FIG. 7 demonstrates the intraomental biologic scaffold supports the engraftment of allogeneic islets under systemic immunosuppression in diabetic rats. A fully MHC-mismatched allogeneic rat transplant combination in which diabetic female Lewis rat (RT1′) (n=4) received 3,000 IEQ WF rat islets (RT1″) in the intraomental biologic scaffold under a protocol of clinically relevant immunosuppressive agents consisting of lymphodepletion induction with anti-lymphocyte serum (0.5 mL i.p. on day −3) and maintenance with mycophenolic acid (MPA) (20 mg/kg/day for days 0-14, then tapered by one-quarter of the dose every 2 days until day 20) and CTLA4Ig (10 mg/kg i.p. on days 0, 2, 4, 6, 8, and 10 and weekly thereafter; abatacept) (arrows). Nonfasting glycemic values for each animal during the follow-up are presented. Graft rejection was defined as return to hyperglycemic state. Each symbol represents an individual animal.

FIGS. 8A-8G demonstrates intraomental allogeneic islet transplantation in a diabetic nonhuman primate. A diabetic cynomolgus monkey received 9,347 IEQ/kg allogeneic islets in the omentum under the cover of clinically relevant immunosuppression therapy. A: Exogenous insulin requirement (EIR) (IU/kg/day), FBG (mg/dL), and PBG. B: Fasting C-peptide (ng/mL) levels measured in the animal over the follow-up period. C-G: Histopathologic pattern of intraomental islet graft on day 49 posttransplant. C: Hematoxylin-eosin staining. D: Immunofluorescence microscopy for the evaluation of immunoreactivity for insulin (INS) (red), GCG (green), and nuclear dye (DAPI) (blue). E: Immunofluorescence for insulin (red) and CD3+ T cells (CD3) (cyan). F and G: Intrainsular neovasculogenesis. F: Immunofluorescence for insulin (red), vascular structure (SMA) (green), and DAPI (blue). G: Immunofluorescence microscopy for insulin (red), endothelial cells (vWF) (green), and DAPI (blue).

FIG. 9A is a graph of the interstitial fluid glucose (mg/dL) as a function of time.

FIG. 9B is a graph of the mean capillary blood glucose (mg/dL) as a function of post-transplant time (days).

FIGS. 10A-10D demonstrate the characterization of endothelial cell-derived exosomes by (A) Nanoparticle tracking analysis and (B) Transmission electron microscopy. (C) Immunofluorescence microscopy detection of HUVEC-derived exosomes (RNA cargo stained in red) uptake on recipient cells MIN6 cells or (D) Human Islets after incubation with HUVEC-derived exosomes.

FIGS. 11A-11B demonstrate islet cell morphology. (A) Human islets were cultured for one week, in normal culture conditions alone or co-cultured with HUVEC cells or HUVEC-derived exosomes. (B) Detailed images after one week in culture of Human islets control (day 7); HUVEC co-cultured islets (day 7); and HUVEC-derived exosomes co-cultured islets (day 7). Pictures are representative of islets morphology as observed by culturing human islets from three independent donors. Magnification 10×.

FIGS. 12A-12C demonstrate islet functionality. (A) Representative images of immunofluorescence staining for insulin (red), glucagon (green), and DAPI (40, 6-diamidino-2-phenylindole; blue) in control islets and Islets treated with HUVEC or HUVEC-derived exosomes. Magnification 32×. (B-C) Glucose stimulated insulin release assay from Human Islets, Human Islets+HUVEC and Human Islets+HUVEC-derived exosomes exposed to 2.2 or 16.6 mmol/I glucose. The level of insulin released to the medium was measured. Results are means±SD of three separate experiments (*** p<0,0001; * p<0.05; ns, non-significant).

FIGS. 13A-13D demonstrate quantitative analysis of the human apoptosis proteome profile array in Human Umbilical Vein Endothelial Cells (HUVEC). Representative images of the expression of the apoptosis proteins in the arrays are shown for the HUVEC (A) and HUVEC-derived exosomes (B). Template showing the exact protein antibody spotted onto the Ray Bio Human apoptosis array kit (C). The results are shown as a bar graph (D), the data were normalized to array-specific positive controls and then compared HUVEC-derived exosomes with HUVEC to establish average fold changes. Any 1.5 fold-increases or s 0.65-fold decreases in signal intensity were considered significant difference in expression.

FIG. 14 is a graph of the stimulation index of islet cells co-cultured with (i) mesenchymal stem cells (MSC), (ii) exosomes derived from MSC, or (iii) MSC-conditioned medium were cultured, or a control.

FIG. 15 is a series of graphs showing the concentration of various inflammation regulators and growth factors expressed by AD-MSC upon stimulation by a low or high dose of TNFα, IFNγ, LPS, or Poly I:C.

### DETAILED DESCRIPTION

The present disclosure provides methods of implanting therapeutic cells in a subject in need thereof. In exemplary embodiments, the method comprises combining the therapeutic cells with a source of a first member of a cell matrix pair to create a therapeutic cell mixture; applying the therapeutic cell mixture to a surface of an organ in the subject; applying a source of a second member of the cell matrix pair to the surface of the organ over the therapeutic cell mixture; and applying a source of the first member of the cell matrix pair to the surface of the organ over the therapeutic cell mixture, thereby forming a therapeutic cell scaffold.

Therapeutic Cells and Therapeutic Agents

As used herein, the term “therapeutic cells” refers to cells that provide therapy or treatment of a disease, disorder, or medical condition, or a symptom thereof, to the subject into whom the cells are implanted. By “implant” is meant to insert, embed, graft, or place into a subject's body. The therapeutic cell can originate from any eukaryote (e.g., animal, mammal, human), from any type of tissue (e.g., lung, brain, heart, skin, endothelial, stem, immune, blood, bone, etc.), and can be of any developmental stage (e.g., embryonic, adult).

In exemplary embodiments of the methods of the present disclosure, the therapeutic cells produce and secrete a therapeutic agent. Examples of therapeutic agents that are contemplated herein include, but are not limited to, natural enzymes, proteins derived from natural sources, recombinant proteins, natural peptides, synthetic peptides, cyclic peptides, antibodies, receptor agonists, cytotoxic agents, immunoglobins, beta-adrenergic blocking agents, calcium channel blockers, coronary vasodilators, cardiac glycosides, antiarrhythmics, cardiac sympathomemetics, angiotensin converting enzyme (ACE) inhibitors, diuretics, inotropes, cholesterol and triglyceride reducers, bile acid sequestrants, fibrates, 3-hydroxy-3-methylgluteryl (HMG)-CoA reductase inhibitors, niacin derivatives, antiadrenergic agents, alpha-adrenergic blocking agents, centrally acting antiadrenergic agents, vasodilators, potassium-sparing agents, thiazides and related agents, angiotensin II receptor antagonists, peripheral vasodilators, antiandrogens, estrogens, antibiotics, retinoids, insulins and analogs, alpha-glucosidase inhibitors, biguanides, meglitinides, sulfonylureas, thizaolidinediones, androgens, progestogens, bone metabolism regulators, anterior pituitary hormones, hypothalamic hormones, posterior pituitary hormones, gonadotropins, gonadotropin-releasing hormone antagonists, ovulation stimulants, selective estrogen receptor modulators, antithyroid agents, thyroid hormones, bulk forming agents, laxatives, antiperistaltics, flora modifiers, intestinal adsorbents, intestinal anti-infectives, antianorexic, anticachexic, antibulimics, appetite suppressants, antiobesity agents, antacids, upper gastrointestinal tract agents, anticholinergic agents, aminosalicylic acid derivatives, biological response modifiers, corticosteroids, antispasmodics, 5-HT₄ partial agonists, antihistamines, cannabinoids, dopamine antagonists, serotonin antagonists, cytoprotectives, histamine H2-receptor antagonists, mucosal protective agent, proton pump inhibitors, H. pylori eradication therapy, erythropoieses stimulants, hematopoietic agents, anemia agents, heparins, antifibrinolytics, hemostatics, blood coagulation factors, adenosine diphosphate inhibitors, glycoprotein receptor inhibitors, fibrinogen-platelet binding inhibitors, thromboxane-A₂ inhibitors, plasminogen activators, antithrombotic agents, glucocorticoids, mineralcorticoids, corticosteroids, selective immunosuppressive agents, antifungals, AIDS-associated infections, cytomegalovirus, non-nucleoside reverse transcriptase inhibitors, nucleoside analog reverse transcriptase inhibitors, protease inhibitors, anemia, Kaposi's sarcoma, aminoglycosides, carbapenems, cephalosporins, glycopeptides, lincosamides, macrolides, oxazolidinones, penicillins, streptogramins, sulfonamides, trimethoprim and derivatives, tetracyclines, antihelmintics, amebicies, biguanides, cinchona alkaloids, folic acid antagonists, quinoline derivatives, Pneumocystis carinii therapy, hydrazides, imidazoles, triazoles, nitroimidzaoles, cyclic amines, neuraminidase inhibitors, nucleosides, phosphate binders, cholinesterase inhibitors, adjunctive therapy, barbiturates and derivatives, benzodiazepines, gamma aminobutyric acid derivatives, hydantoin derivatives, iminostilbene derivatives, succinimide derivatives, anticonvulsants, ergot alkaloids, antimigrane preparations, biological response modifiers, carbamic acid eaters, tricyclic derivatives, depolarizing agents, nondepolarizing agents, neuromuscular paralytic agents, CNS stimulants, dopaminergic reagents, monoamine oxidase inhibitors, COMT inhibitors, alkyl sulphonates, ethylenimines, imidazotetrazines, nitrogen mustard analogs, nitrosoureas, platinum-containing compounds, antimetabolites, purine analogs, pyrimidine analogs, urea derivatives, antracyclines, actinomycinds, camptothecin derivatives, epipodophyllotoxins, taxanes, vinca alkaloids and analogs, antiandrogens, antiestrogens, nonsteroidal aromatase inhibitors, protein kinase inhibitor antineoplastics, azaspirodecanedione derivatives, anxiolytics, stimulants, monoamind reuptake inhibitors, selective serotonin reuptake inhibitors, antidepressants, benzisooxazole derivatives, butyrophenone derivatives, dibenzodiazepine derivatives, dibenzothiazepine derivatives, diphenylbutylpiperidine derivatives, phenothiazines, thienobenzodiazepine derivatives, thioxanthene derivatives, allergenic extracts, nonsteroidal agents, leukotriene receptor antagonists, xanthines, endothelin receptor antagonist, prostaglandins, lung surfactants, mucolytics, antimitotics, uricosurics, xanthine oxidase inhibitors, phosphodiesterase inhibitors, metheamine salts, nitrofuran derivatives, quinolones, smooth muscle relaxants, parasympathomimetic agents, halogenated hydrocarbons, esters of amino benzoic acid, amides (e.g., lidocaine, articaine hydrochloride, or bupivacaine hydrochloride), antipyretics, hypnotics and sedatives, cyclopyrrolones, pyrazolopyrimidines, nonsteroidal anti-inflammatory drugs, opioids, para-aminophenol derivatives, alcohol dehydrogenase inhibitor, heparin antagonists, adsorbents, emetics, opioid antagonists, cholinesterase reactivators, nicotine replacement therapy, vitamin A analogs and antagonists, vitamin B analogs and antagonists, vitamin C analogs and antagonists, vitamin D analogs and antagonists, vitamin E analogs and antagonists, vitamin K analogs and antagonists.

The therapeutic agent can be a peptide, blood factor, hormone, growth factor, cytokine, lymphokine, or other hematopoietic factor, including, but not limited to: M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, TNFα, TNF1, TNF2, G-CSF, Meg-CSF, GM-CSF, thrombopoietin, stem cell factor, and erythropoietin. Additional growth factors for use herein include angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor α, cytokine-induced neutrophil chemotactic factor 1, cytokine-induced neutrophil, chemotactic factor 2α, cytokine-induced neutrophil chemotactic factor 2β, β endothelial cell growth factor, endothelin 1, epithelial-derived neutrophil attractant, glial cell line-derived neutrophic factor receptor α 1, glial cell line-derived neutrophic factor receptor α 2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, pre-B cell growth stimulating factor, stem cell factor, stem cell factor receptor, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor binding protein III, tumor necrosis factor receptor type I, tumor necrosis factor receptor type II, urokinase-type plasminogen activator receptor, and chimeric proteins and biologically or immunologically active fragments thereof. In exemplary aspects, the therapeutic agent is insulin or an insulin analog. As used herein, the term “insulin analog” means a non-naturally occurring molecule that is designed to mimic the actions of insulin. In exemplary aspects, the insulin analog is structurally similar to insulin. In exemplary aspects, the insulin analog is a rapid acting insulin analog, long acting insulin analog, or a pre-mixture of insulin analogs. Such insulin analogs are known in the art and include, for example, Aspart, Glulisine, Lyspro, Humalog®, NovoRapid®, Lantus®, Levemir®, Tresiba, Humalog Mix 25, Humalog Mix 50@, and NovoMix 30@. The insulin analog may be a fused insulin analog, e.g., Albulin. See, e.g, Duttaroy et al., Diabetes 54(1): 251-258 (2005).

In exemplary aspects, the therapeutic cells are pancreatic islet cells, induced pluripotent stem cells (iPSC), embryonic stem cells, or cells modified to produce and secrete insulin or an insulin analog. Cells modified to produce and secrete insulin or an insulin analog are known in the art. See, e.g., WO2016/179106, CN102051372, CN1668324, CN101160390, US20040191901, CN101724602, U.S. Pat. No. 7,056,734, US20140234269, US20060281174, US20140256043, CN101432425, US20140242038. In exemplary aspects, the therapeutic cells are pancreatic islet cells produced from iPSCs. See, e.g., Mihara et al., J Tissue Eng Regen Med (March 2017); doi 10.1002/term.2228; Yabe et al., J Diabetes 9(2): 168-179 (2017); Walczak et al., J Transl Med 14(1): 341 (2016); Rajaei et al., J cell Physiol doi: 10.1002/jcp.25459; and Enderami et al., J Cell Physiol (December 2016); doi: 10.1002jcp.25721.

In exemplary aspects, the therapeutic cells are pancreatic islet cells. In exemplary instances, the pancreatic islet cells are human pancreatic islet cells, optionally, human pancreatic islet cells which are allogeneic to the subject of the methods of the present disclosure. In some aspects, the pancreatic islet cells are isolated from a human cadaver or other human donor. Methods of isolating such cells from a human cadaver are described in the art. See, e.g., Halberstadt et al., Methods Mol Biol 1001: 227-259 (2013). In exemplary aspects, the pancreatic islet cells are non-human pancreatic islet cells, such as those described in Ranuncoli et al., Cell Transplant 9(3): 409-414 (2000); Dufrane et al., Xenotransplantation 13(3): 204-214 (2006).

In some instances, the islet cells are part of an islet, such that the methods of implanting therapeutic cells in a subject of the present disclosure encompass a method of implanting an islet. In some aspects, the method comprises combining the islet with a source of a first member of a cell matrix pair to create a islet mixture; applying the islet mixture to a surface of an organ in the subject; applying a source of a second member of the cell matrix pair to the surface of the organ over the islet mixture; and applying a source of the first member of the cell matrix pair to the surface of the organ over the islet mixture, thereby forming a therapeutic cell scaffold.

In some aspects, the therapeutic cells are part of a cell cluster. In exemplary aspects, the therapeutic cells are pancreatic islet cell clusters, induced pluripotent stem cell (iPSC) clusters, embryonic stem cell clusters, or clusters of cells modified to produce and secrete insulin or an insulin analog.

Cell Matrix Pairs

As used herein, the term “cell matrix pair” refers to a pair of molecules (e.g., a first member and a second member) that are involved in the extracellular matrix (ECM) providing support to cells. A cell matrix pair may comprise components of the ECM, including, e.g., proteoglycans, nonproteoglycan polysaccharides and proteins. The cell matrix pair may comprise, e.g., glycosaminoglycans (GAGs), heparin sulfate, chondroitin sulfate, karatan sulfate, collagen, elastin, fibronectin, laminin, integrin, or a precursor form thereof. In exemplary aspects, the cell matrix pair comprises fibronectin and collagen. In exemplary aspects, the cell matrix pair comprises laminin and collagen or nidogen. In exemplary aspects, the cell matrix pair comprises thrombin and fibrinogen.

In exemplary embodiments of the methods of the present disclosure, a source of a first member of a cell matrix pair is combined with the therapeutic cells to create a therapeutic cell mixture. In exemplary aspects, the first member of the cell matrix pair is fibronectin and the source of fibronectin is plasma. In exemplary aspects, the plasma is autologous to the subject of the method. In this regard, the method in some aspects comprises collecting plasma from the subject prior to applying the therapeutic cells to a surface of the organ in the subject. Such methods comprising collecting plasma is further described herein. See “Additional Steps.” In exemplary aspects, the source of the second member of the cell matrix pair is recombinant human thrombin and the source of the first member of the cell matrix pair is plasma.

Implantation Sites

With regard to the methods of the present disclosure, therapeutic cells are implanted into the subject at a particular site, i.e., an implantation site. In exemplary aspects, the therapeutic cells, e.g., as part of a therapeutic cell mixture, are applied at an implantation site. In various embodiments, the implantation site is an organ. For example, in exemplary aspects, the therapeutic cells, e.g., as part of a therapeutic cell mixture, are applied to a surface of an organ in the subject. In exemplary instances the organ is a mesothelial organ, e.g., comprises mesothelial tissue. In exemplary aspects, the mesothelial organ comprises or is a peritoneum. In exemplary embodiments, the organ is aliver, pancreas, or an organ of the gastrointestinal tract (e.g., oesophagus, pharynx, mouth, stomach, omentum, small intestine (duodenum, jejunum, ileum), large intestine (e.g., cecum, colon), rectum, anus, liver, gall bladder, pancreas, salivary gland, lips, teeth, tongue, or epiglottis). In exemplary aspects, the organ or implantation site is a gastric submucosa, genitourinary tract, muscle, bone marrow, kidney (e.g., kidney capsule), anterior eye chamber, testis, or thymus.

In exemplary instances, the therapeutic cells, e.g., as part of a therapeutic cell mixture, are applied to a surface of an omentum, e.g., the greater omentum or the less omentum. The omentum is composed of two mesothelial sheets containing rich capillary networks draining into the portal venous system. It is easy to mobilize and large enough to accommodate islet grafts (300-1,500 cm² surface area in humans). In exemplary aspects, the method comprises laparoscopically layering the therapeutic cell mixture along an axis of the omentum. As used herein, the term “laparascopically” refers to a surgical procedure in which a fiber-optic instrument is inserted through the abdominal wall to view the organs in the abdomen or to permit a surgical procedure. In exemplary aspects, the method further comprises folding opposing sides of the omentum along the axis to envelope the therapeutic cell scaffold in the omentum. In exemplary embodiments of the methods of the present disclosure, the method comprises combining the therapeutic cells with plasma to form a therapeutic cell mixture, applying the therapeutic cell mixture to the surface of the omentum of the subject, applying thrombin over the therapeutic cell mixture, and applying plasma over the therapeutic cell mixture and the thrombin.

Subjects

In exemplary embodiments, the subject of the methods of the present disclosures is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In exemplary aspects, the mammal is a human.

In exemplary instances, the mammal is a human which has a deficiency in the therapeutic agent provided by the implanted therapeutic cells. In exemplary aspects, the therapeutic cells produce and secrete insulin, or an analog thereof. In exemplary aspects, the subject suffers from a metabolic disease, e.g., diabetes or obesity. In exemplary instances, the diabetes is Type I diabetes, and in particular aspects, the Type I diabetes occurs with severe hypoglycemia. In exemplary aspects, the subject is treated for the metabolic disease with exogenous insulin therapy. In exemplary aspects, the subject is one who, prior to implantation with therapeutic cells, was treated for the metabolic disease with exogenous insulin therapy. In exemplary instances, the implanted therapeutic cells provides the needed insulin such that exogenous insulin therapy may stop after implantation. According, in some aspects, the methods of the present disclosure comprise ceasing exogenous insulin therapy after the therapeutic cells are implanted into the subject.

Supporting Cellular Composition

In exemplary embodiments, the method of the present disclosure is a method of implanting pancreatic islet cells in a subject, comprising co-administering therapeutic cells, e.g., pancreatic islet cells, and a supporting cellular composition. In exemplary aspects, the therapeutic cells, e.g., pancreatic islet cells, and supporting cellular composition are admixed or combined prior to implantation into the subject and then the admixture is implanted simultaneously. In exemplary aspects, the therapeutic cells, e.g., pancreatic islet cells, and supporting cellular composition are implanted sequentially into the subject, e.g., the therapeutic cells, e.g., pancreatic islet cells, are implanted before implantation of the supporting cellular composition, or the therapeutic cells, e.g., pancreatic islet cells, are implanted after implantation of the supporting cellular composition.

In exemplary aspects, the therapeutic cells, e.g., pancreatic islet cells, are admixed with the supporting cell composition prior to implantation and the method further comprises combining the admixture of cells with a source of a first member of a cell matrix pair to create a therapeutic cell mixture. The method comprises applying the therapeutic cell mixture to a surface of an organ in the subject; and applying a source of a second member of the cell matrix pair to the surface of the organ over the therapeutic cell mixture thereby forming a therapeutic cell scaffold. In exemplary aspects, the method further comprises applying a source of the first member of the cell matrix pair to the surface of the organ over the therapeutic cell mixture. In exemplary instances, the first member of the cell matrix pair is fibronectin and the source of fibronectin is plasma, e.g., autologous plasma. In exemplary aspects, the second member of the cell matrix pair is thrombin and the source of thrombin is a recombinant human thrombin. In exemplary aspects, the method further comprises applying plasma to the surface of the organ over the therapeutic cell mixture, e.g., applying plasma to the surface of the organ after applying the thrombin.

As used herein, the term “supporting cellular composition” refers to a composition comprising cells which support the viability and/or function of the therapeutic cells, e.g., pancreatic islet cells. In exemplary aspects, the supporting cellular composition aids in the overall engraftment of the therapeutic cells, e.g., pancreatic islet cells, into the subject. In exemplary aspects, the supporting cellular composition assists in protecting the therapeutic cells, e.g., pancreatic islet cells, from elimination from the subject or protects the therapeutic cells, e.g., pancreatic islet cells, from an immune response. In exemplary aspects, the supporting cellular composition increases the viability and/or survival of the therapeutic cells, e.g., pancreatic islet cells, by increasing the oxygenation and/or vascularization of the cells or by increasing access to nutrients required by the cells. In exemplary aspects, the supporting cellular composition increases or maintains the production and/or secretion of therapeutic agent, e.g., insulin by the therapeutic cells, e.g., pancreatic islet cells. In exemplary aspects, the supporting cellular composition comprises endothelial-derived exosomes or endothelial cells. In additional or alternative aspects, the supporting cellular composition comprises immunomodulatory cells, stem cells, micronized fat cell clusters, bone marrow-derived mononuclear cells, cells of an adipose-derived stromal vascular fraction, endothelial progenitor cells, pericytes, hematopoietic progenitor cells, monocytes, leukocytes, fibroblastic reticular stromal cells, a fat tissue fraction comprising mesenchymal stem cells (MSC), or cell mixtures thereof. In additional or alternative aspects, the supporting cellular composition comprises exosomes derived from any one or more of the immunomodulatory cells, stem cells, micronized fat cell clusters, bone marrow-derived mononuclear cells, cells of an adipose-derived stromal vascular fraction, endothelial progenitor cells, pericytes, hematopoietic progenitor cells, monocytes, leukocytes, fibroblastic reticular stromal cells, or MSC. In exemplary aspects, the immunomodulatory cells are Treg cells. In exemplary aspects, the stem cells are MSC, bone marrow-derived stem cells, adipose tissue-derived stem cells, e.g., AD-MSC, endothelial progenitor cells, or cell mixtures thereof.

In exemplary aspects, the supporting cellular composition comprises MSC or exosomes derived from MSC. In exemplary aspects, the MSC are adipose-derived MSC (AD-MSC). In exemplary aspects, the MSC are positive for cell surface expression of CD146. In exemplary aspects, the method of implanting pancreatic islet cells in a subject, comprises pre-selecting for MSC expressing CD146. In exemplary aspects, the method of implanting pancreatic islet cells in a subject, comprises culturing MSC under conditions which induce CD146 expression. In exemplary instances, the method comprises culturing MSC in the presence of IFNγ or TNFα to yield a population of MSC expressing CD146. In exemplary aspects, the method comprises culturing MSC with about 5 ng to about 100 ng IFNγ or TNFα for about 1-7 days, purifying MSC positive for expression of CD146, and then co-administering pancreatic islet cells with the purified CD146+MSC to a subject. In exemplary aspects, the method comprises culturing MSC with about 5 ng to about 50 ng IFNγ or TNFα for about 2-3 days. In exemplary aspects, the method comprises culturing MSC with about 5 ng to about 25 ng (e.g., about 8 ng to about 15 ng, about 10 ng) IFNγ or TNFα for about 2 days.

In exemplary aspects, the supporting cell composition comprises a heterogeneous cell population comprising different cell types. For example, in exemplary aspects, the supporting cell composition comprising bone-marrow derived mononuclear cells (BM-MNC) and an adipose tissue-derived stromal vascular fraction, within which MSCs are mixed with endothelial progenitor cells (EPC), pericytes, hematopoietic progenitors, monocytes, and leukocytes. Also, for example, the supporting cell composition can comprise micronized fat or micronized fat cell clusters. In exemplary instances, the supporting cell composition comprises microfragmented adipose tissue with intake stromal cells and MSCs. In exemplary aspects, the supporting cell composition comprises refined fat tissue produced via a Lipogems® technique, which washes, emulsifies, and rinses adipose tissue. The resulting fraction comprises adipose clusters of about 0.3 to about 0.8 mm. The Lipogems® produced fraction can comprise pericytes within an intact stromal vascular component. The Lipogems® technique, the adipose fraction produced thereby, and a variety of applications thereof, are described in Tremolada et al., Curr Stem Cell Rep 2: 304-312 (2016).

In exemplary aspects, the supporting cell composition comprises a homogeneous cell population comprising substantially the same cell types. For example, in exemplary aspects, the supporting cell composition comprises culture-expanded adult stem cells from BM-MNC or adipose tissue stem cells (ADSC). Also, for example, the supporting cell composition can comprise fibroblastic reticular stromal cells, e.g., fibroblastic reticular stromal cells isolated from lymph nodes or pancreas.

In exemplary aspects, the supporting cell composition comprises pre-selected adult stem cells with enhanced immunomodulatory properties, e.g., adult stem cells having positive expression of the cell surface marker CD146.

Targeting Molecules

In exemplary embodiments, the method of the present disclosure is a method of implanting pancreatic islet cells in a subject, comprising combining pancreatic islet cells with a targeting molecule prior to implantation into the subject to create a therapeutic cell mixture. In exemplary aspects, the pancreatic islet cells are further combined with source of a first member of a cell matrix pair. In exemplary aspects, the pancreatic islet cells are further combined with a supporting cellular composition. The method comprises applying the therapeutic cell mixture to a surface of an organ in the subject; and applying a source of a second member of the cell matrix pair to the surface of the organ over the therapeutic cell mixture thereby forming a therapeutic cell scaffold. In exemplary aspects, the method further comprises applying a source of the first member of the cell matrix pair to the surface of the organ over the therapeutic cell mixture. In exemplary instances, the first member of the cell matrix pair is fibronectin and the source of fibronectin is plasma, e.g., autologous plasma. In exemplary aspects, the second member of the cell matrix pair is thrombin and the source of thrombin is a recombinant human thrombin. In exemplary aspects, the method further comprises applying plasma to the surface of the organ over the therapeutic cell mixture, e.g., applying plasma to the surface of the organ after applying the thrombin.

As used herein, the term “targeting molecule” refers to a molecule or agent that specifically recognizes and binds to a binding partner, such that the targeting molecule directs the delivery of a compound to the target: pancreatic islet cells. Targeting molecules include, but are not limited to, antibodies and antigens, biotin and streptavidin, ligands and receptors, and the like. In exemplary aspects, the targeting molecule is directly attached to the pancreatic islet cells. In alternative aspects, the targeting molecule is indirectly attached to the pancreatic islet cells. In some aspects, the targeting molecule is encapsulated with the pancreatic islet cells.

Encapsulation

In exemplary aspects, the therapeutic cells of the methods of the present disclosure are not encapsulated or are “naked”. In alternative exemplary instances, the therapeutic cells are encapsulated or comprise a coating. In exemplary aspects, the therapeutic cells are microencapsulated, macroencapsulated, nanoencapsulated or conformally coated. Methods of encapsulating and conformally coating therapeutic cells, e.g., pancreatic islet cells, are known in the art. See, e.g., U.S. Patent Application Publication 2014/0147483; Tomei et al., Expert Opin Biol Ther 15(9): (2015); Tomei et al., PNAS 111(29): 10514-10519 (2014); Scharp et al., Adv Drug Deliv Rev 67-68:65-73 (2014); Basta et al., Curr Diab Rep 11(5): 384-391 (2011).

In exemplary aspects, the therapeutic cells are encapsulated with the supporting cell composition. In exemplary aspects, the therapeutic cells are encapsulated separately from the supporting cell composition. In exemplary aspects, the therapeutic cells are encapsulated with a targeting molecule. In exemplary aspects, the therapeutic cells are encapsulated separately from a targeting molecule. Methods of encapsulating islet cells and conformally coating islet cells are described in the art. See, e.g., WO2010078202; Tomei et al., (2014) supra.

In exemplary aspects, the therapeutic cells, e.g., pancreatic islet cells, are encapsulated within a covalently stabilized coating formed by reacting (i) alginate-[polyalkylene glycol (PAG)-X¹]ₙ, and (ii) multi-functional PAG-X², to form covalent bonds, wherein n is an integer greater than 0, a first one of X¹ and X² is N₃, and a second one of X¹ and X² is selected from the group consisting of:

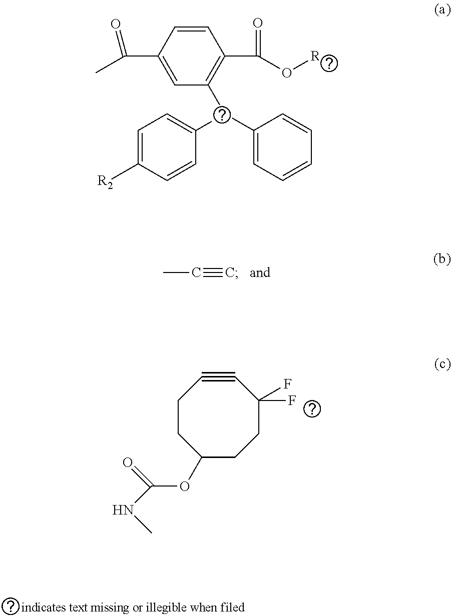

wherein R¹═CH₃, CH₂CH₃, CH(CH₃)₂, or

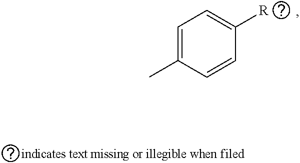

R²═N(CH₃)₂, OCH₃, OH, CH₃, H, F, Cl, Br or NO₂, and R³═OCH₃, CH₃, H, F, Cl or NO₂.

In exemplary aspects, the covalently stabilized coating includes a plurality of monolayers. In exemplary aspects, the plurality of monolayers alternate between monolayers of Alginate-[PAG-X¹], reaction products and monolayers of multi-functional PAG-X² reaction products. In exemplary instances, the multi-functional PAG-X2 comprises a multi-arm PAG-X² having at least three PAG-X² arms. In exemplary aspects, the alginate-[polyethylene glycol (PAG)-X¹], molecule, the multi-functional PAG-X2 molecule, or both, include an additional terminal ligand, X³. In some aspects, the additional terminal ligand, X³, is selected from the group consisting of proteins, imaging labels, nanoparticles, biopolymers, RNA, DNA, and fragments of RNA or DNA.

In exemplary aspects, the therapeutic cells, e.g., pancreatic islet cells, are conformally coated with a coating material. In exemplary instances, the therapeutic cells, e.g., pancreatic islet cells, are conformally coated by: a) injecting a water phase within a coaxial oil phase in a coating device that allows for a transition from dripping to jetting and flow elongation of the water phase within the oil phase; b) adding the therapeutic cells, e.g., pancreatic islet cells, and the coating material to the water phase, wherein polymerization of the coating material occurs downstream of breakup of the water phase jet into particles, resulting in the conformal coating of the therapeutic cells, e.g., pancreatic islet cells, with the coating material. See, e.g., U.S. Patent Application Publication No. 2014/0147483, the contents of which is incorporated by reference in its entirety.

In exemplary aspects, the method of conformally coating the therapeutic cells comprises c) collecting the outflow of the coating device; d) purifying the conformally coated pancreatic islet cells and pancreatic islet cells-free coating material from the oil phase; e) separating the conformally coated pancreatic islet cells from the pancreatic islet cells-free coating material, or f) a combination thereof. In exemplary aspects, the step of purifying the conformally coated pancreatic islet cells and pancreatic islet cells-free coating material from the oil phase comprises the steps of: a) centrifuging the coating device outflow to separate the oil phase from the conformally coated pancreatic islet cells and pancreatic islet cells-free coating material; and b) optionally performing hexane extraction until the oil phase is completely eliminated from the conformally coated pancreatic islet cells and pancreatic islet cells-free coating material. In exemplary aspects, the step of separating the conformally coated pancreatic islet cells from the pancreatic islet cells-free coating material is performed by gradient centrifugation or by settlement of coated clusters by gravity. In exemplary aspects, greater than 95% of the conformally coated pancreatic islet cells is purified from the pancreatic islet cells-free coating material.

In exemplary aspects, the coating material comprises polyethylene glycol (PEG). In exemplary aspects, the water phase comprises about 75,000 islet cells/ml, and further comprises: a) about 10% PEG, about 2% Pluronic-F68, and about 0.62% w/v DTT in serum-free media at pH 6-7; b) about 5% PEG, about 1% Pluronic-F68, and about 0.31% w/v DTT in HBSS at pH 6-7; c) about 5% PEG, about 1% Pluronic-F68, about 0.8% medium viscosity G-groups alginate and about 0.31% w/v DTT in HBSS without Ca²⁺ and Mg²⁺ at pH 6-7; or d) about 5% PEG, about 0.8% medium viscosity G-groups alginate and about 0.31% w/v DTT in HBSS without Ca²⁺ and Mg²⁺ at pH 6-7. In exemplary instances, the oil phase comprises polypropylene glycol (PPG) with about 10% Span80, wherein said oil phase optionally further comprises about 0.02 or about 0.2% triethanolamine.

In exemplary aspects, the coating device comprises a flow chamber comprising a flow focusing region and a channel downstream of the flow focusing region, and wherein the flow chamber has at least one of the following properties: a) the diameter of the inner channel of the flow focusing region is restricted from 10 d to d along its length, wherein d is about 1 mm; b) the focusing angle of the flow focusing region is about 60 degrees; c) the channel downstream of the flow focusing region is about 1 mm in diameter; and d) the water phase is injected into the flow chamber through a needle or a catheter whose tip is localized about 0.5 mm upstream of the focusing region.

In exemplary aspects, the process used to conformally coat the pancreatic islet cells has at least one of the following properties: a) the ratio of the oil phase velocity to the water phase velocity is about 350; b) the ratio of the oil phase viscosity to the water phase viscosity is about 3.5, 13, or 130; c) the water phase is injected into the oil phase first at about 50 μl/min and then reduced to about 10 μl/min, while the oil phase is maintained at about 3.5 ml/min; and d) air is injected into the oil phase before injection of the water phase.

In exemplary aspects, the water phase comprises 5% PEG, 0.8% MVG, 75,000 islet cells/ml, and 0.31% w/v DTT in HBSS without Ca²⁺ and Mg²⁺ at pH 6-7; wherein said oil phase comprises PPG with 10% Span80, wherein said oil phase optionally comprises 0.02% or 0.2% triethanolamine; and wherein said coating device comprises a flow chamber comprising a flow focusing region with a channel whose inner diameter is reduced from 10 mm to 1 mm with a focusing angle of 60 degrees, and a channel downstream of the flow focusing region that is about 1 mm in diameter. In exemplary aspects, the method comprises the steps of: a) applying the oil phase to the flow chamber; b) optionally injecting air into the flow chamber through a catheter whose tip is localized 0.5 mm upstream of the base of the focusing region; and c) injecting the air, if present, and the water phase into the flow chamber through said catheter, wherein the water phase is first injected at 50 μl/min and then reduced to 10 μl/min, while the oil phase is maintained at 3.5 ml/min, such that the surface tension between the water and the oil phase causes the water jet to break up into microliter droplets comprising the conformally coated pancreatic islet cells and pancreatic islet cells-free coating material. In exemplary aspects, the method used to conformally coat the pancreatic islet cells further comprises the steps of: d) collecting the outflow from the flow chamber; e) centrifuging the outflow to separate the conformally coated pancreatic islet cells and pancreatic islet cells-free coating material from the oil phase; f) removing the oil phase supernatant from the conformally coated pancreatic islet cells and pancreatic islet cells-free coating material; g) resuspending the conformally coated pancreatic islet cells and pancreatic islet cells-free coating material in a composition comprising hexane; h) centrifuging the mixture of step g) to separate the conformally coated pancreatic islet cells and pancreatic islet cells-free coating material from the hexane; i) removing the hexane supernatant; j) resuspending the conformally coated pancreatic islet cells and pancreatic islet cells-free coating material in a composition comprising hexane and a buffer (without Ca²⁺ and Mg²⁺); k) centrifuging the mixture of step j) to separate the conformally coated pancreatic islet cells and pancreatic islet cells-free coating material from the hexane and buffer (without Ca²⁺ and Mg²⁺); l) removing the hexane/buffer supernatant; and m) resuspending the conformally coated pancreatic islet cells and pancreatic islet cells-free coating material in buffer containing Ca²⁺ and Mg²⁺. In exemplary aspects, the method further comprises the steps of: n) layering solutions to form a density gradient capable of separating the conformally coated pancreatic islet cells and the pancreatic islet cells-free coating material; o) applying the conformally coated pancreatic islet cells and pancreatic islet cells-free coating material to the density gradient; p) centrifuging the density gradient to separate the conformally coated pancreatic islet cells from the pancreatic islet cells-free coating material; and q) removing the supernatant containing the pancreatic islet cells-free coating material.

In exemplary aspects, the conformal coating around the pancreatic islet cells ranges from 10-20 μm in thickness. In exemplary aspects, greater than 90% of the pancreatic islet cells introduced into the coating device is conformally coated.

Additional Steps

With regard to the methods of the present disclosure, the methods may include additional steps. For example, the method of implanting therapeutic cells in a subject may include repeating one or more of the recited step(s) of the method. Accordingly, in exemplary aspects, the method comprises repeating the step of applying to a surface of an organ in the subject therapeutic cells of a therapeutic cell mixture created by combining the therapeutic cells with a source of a first member of a cells matrix pair. In exemplary aspects, the method comprises re-applying the therapeutic cell mixture every 6 to 12 months, or as needed based on the subject's blood glucose, Hemoglobin A1C (HA1C) levels, C peptide levels, HOMA indices, or BETA scores and/or frequency of hypoglycemic episodes. Alternatively or additionally, the method may comprise repeating the steps of applying a source of a second member or of a first member of the cell matrix pair.

In exemplary embodiments, wherein either the first member or the second member of the cell matrix pair is a plasma protein and the source thereof is plasma, the method of implanting therapeutic cells in a subject further comprises collecting plasma from the subject. In this regard, the plasma is autologous to the subject receiving the therapeutic cells. In exemplary aspects, the plasma is conditioned plasma. In certain aspects, the method further comprises administering to the subject one or more of an omega-3 fatty acid, an anti-inflammatory agent, an immunomodulatory molecule, a growth factor, or a combination thereof, prior to collecting plasma from the subject, followed by collecting the plasma from the subject. In exemplary instances, the plasma is processed plasma. In certain aspects, the plasma has been enriched for one or more characteristics or expression of a particular protein or molecule present in the plasma, or the plasma has been processed to remove certain proteins or molecules. In exemplary aspects, the plasma has been processed for enrichment in one or more of a platelet-rich plasma (PRP), anti-inflammatory factor, pro-angiogenic factor, growth factor, or a combination thereof.

The methods of implanting therapeutic cells can comprise yet further steps before or after the application of the therapeutic cell mixture, or both. In exemplary aspects, the methods of implanting therapeutic cells of the present disclosure comprises reducing the subject's intake of exogenous insulin and/or other anti-diabetes drugs. In exemplary aspects, the methods further comprise preparing the therapeutic cells by, e.g., genetically engineering cells with a vector comprising a nucleic acid encoding a therapeutic agent. In exemplary aspects, the methods further comprise culturing the genetically engineered cells. In exemplary embodiments, the methods of implanting therapeutic cells comprises preparing the therapeutic cells by culturing the cells in the presence of endothelial-derived exosomes or endothelial cells or the contents of such exosomes. Accordingly, the present disclosure provides a method of implanting cells comprising one or more steps to prepare the therapeutic cells for implantation into the subject.

Methods of Preparing Islets

The present disclosure provides a method of preparing pancreatic islet cells for implantation into a subject. In exemplary embodiments, the method comprises culturing the pancreatic islet cells with endothelial-derived exosomes or endothelial cells (EC) or the contents of endothelial-derived exosomes. In exemplary aspects, the endothelial cells are human umbilical vein endothelial cells (HUVEC) or the endothelial-derived exosomes are HUVEC-derived endosomes. In exemplary aspects, the pancreatic islet cells are human pancreatic islet cells. In exemplary instances, about 100-500 pancreatic islet cells are cultured with about 10⁴ to about 10⁶ EC or about 10¹⁰ to about 10¹⁵ EC-derived exosomes, per ml. In exemplary aspects, about 1000-10,000 (e.g., 1000-5000) pancreatic islet cells are cultured with about 104 to about 10⁶ endothelial cells or about 10¹⁰ to about 10¹⁵ exosomes, per ml. In exemplary instances, about 100 IEQ to about 1000 IEQ pancreatic islet cells are cultured with about 100 EC to about 1000 EC or about 1 to about 100 ug/ml EC-derived exosomes. In exemplary instances, about 250 IEQ to about 750 IEQ pancreatic islet cells are cultured with about 250 EC to about 750 EC or about 5 to about 50 ug/ml EC-derived exosomes. In exemplary instances, about 400 to about 600 IEQ pancreatic islet cells are cultured with about 400 EC to about 600 EC or about 5 to about 15 ug/ml EC-derived exosomes. In exemplary instances, about 500 IEQ pancreatic islet cells are cultured with about 500 EC or about 10 ug/ml EC-derived exosomes. In some aspects, the methods of preparing pancreatic islet cells comprises culturing the pancreatic islet cells with endothelial-derived exosomes or endothelial cells for at least about 24 to about 48 hours. Optionally, the method comprises culturing the pancreatic islet cells with endothelial-derived exosomes or endothelial cells for at least about 3 to 6 days or for at least about 1 to about 2 weeks.

In exemplary embodiments, the method of preparing pancreatic islet cells for implantation into a subject, comprises culturing the pancreatic islet cells with contents of endothelial-derived exosomes prior to implanting the pancreatic islet cells into the subject. In exemplary aspects, the method of preparing pancreatic islet cells for implantation into a subject, comprises culturing the pancreatic islet cells with hepatocyte growth factor (HGF), thrombospondin-1 (TSP-1), a laminin, a collagen, insulin growth factor binding protein-1 (IGFBP-2), CD40, IGFBP-1, sTNFRII, CD40L, TNFα, cIAP-2, IGFBP-3, TNFβ, CytoC, IGFBP-4, TRAIL R1, TRAIL R2, TRAIL R3, bad, IGF-1 sR, TRAIL R4, HSP60, p27, Caspase 8, IGF-2, or a combination thereof, prior to implanting the pancreatic islet cells into the subject.

The present disclosure also provides a method of preparing pancreatic islet cells for implantation into a subject, comprising culturing the pancreatic islet cells with MSC-derived exosomes or MSCs or the contents of MSC-derived exosomes. In exemplary aspects, the MSC are adipose-derived MSC (AD-MSC). In exemplary aspects, the MSC are positive for expression of CD146. In exemplary aspects, the pancreatic islet cells are human pancreatic islet cells. In exemplary instances, about 100-500 pancreatic islet cells are cultured with about 10⁴ to about 10⁶ MSC or about 10¹⁰ to about 10¹⁵ exosomes, per ml. In exemplary instances, about 100 IEQ to about 1000 IEQ pancreatic islet cells are cultured with about 100 MSC to about 1000 MSC or about 1 to about 100 ug/ml exosomes. In exemplary instances, about 250 IEQ to about 750 IEQ pancreatic islet cells are cultured with about 250 MSC to about 750 MSC or about 5 to about 50 ug/ml exosomes. In exemplary instances, about 400 to about 600 IEQ pancreatic islet cells are cultured with about 400 MSC to about 600 MSC or about 5 to about 15 ug/ml exosomes. In exemplary instances, about 500 IEQ pancreatic islet cells are cultured with about 500 MSC or about 10 ug/ml exosomes. In some aspects, the methods of preparing pancreatic islet cells comprises culturing the pancreatic islet cells with MSC-derived exosomes or MSC for at least about 24 to about 48 hours. Optionally, the method comprises culturing the pancreatic islet cells with MSC-derived exosomes or MSC for at least about 3 to 6 days or for at least about 1 to about 2 weeks.

In exemplary aspects, the MSCs are pre-selected for expression of a cell surface marker. In exemplary instances, the cell surface marker is CD146. In exemplary aspects, the method of preparing islets for implantation into a subject comprises culturing MSC under conditions which induce CD146 expression. In exemplary instances, the method comprises culturing MSC in the presence of IFNγ or TNFα to yield a population of MSC expressing CD146. In exemplary aspects, the method comprises culturing MSC with about 5 ng to about 100 ng IFNγ or TNFα for about 1-7 days, purifying MSC positive for expression of CD146, and then co-administering pancreatic islet cells with the purified CD146+MSC to a subject. In exemplary aspects, the method comprises culturing MSC with about 5 ng to about 50 ng IFNγ or TNFα for about 2-3 days. In exemplary aspects, the method comprises culturing MSC with about 5 ng to about 25 ng (e.g., about 8 ng to about 15 ng, about 10 ng) IFNγ or TNFα for about 2 days.

In exemplary embodiments, the method of preparing pancreatic islet cells for implantation into a subject, comprises culturing the pancreatic islet cells with contents of MSC-derived exosomes prior to implanting the pancreatic islet cells into the subject. In exemplary aspects, the method of preparing pancreatic islet cells for implantation into a subject, comprises culturing the pancreatic islet cells with IGFB-1, IGFB-2, IGFB-3, IGFB-4, IGFB-6, IGF-1, IGF-1 SR, IGF-II, M-CSF, MCSF R, PDGF-AA, VEGF, IL-6, IL-8, Eotaxin, ICAM-1, IFNγ, CCL, MCP-2, MIP-1α, RANTES, TNFα, or a combination thereof, prior to implanting the pancreatic islet cells into the subject.

In various aspects, the disclosure provides a method of implanting pancreatic islet cells in a subject, comprising: preparing the pancreatic islet cells according to the methods of preparing cells as described herein; combining pancreatic islet cells prepared in step (a) with a source of a first member of a cell matrix pair to create a therapeutic cell mixture; applying the therapeutic cell mixture to a surface of an organ in the subject; and applying a source of a second member of the cell matrix pair to the surface of the organ over the therapeutic cell mixture, thereby forming a therapeutic cell scaffold.

Compositions

The present disclosure further provides compositions comprising therapeutic cells in combination with one or more of: a source of a first member of a cell matrix pair, a second member of a cell matrix pair, and a supporting cellular composition. In exemplary aspects, the composition is a pharmaceutical composition comprising therapeutic cells in combination with one or more of: a source of a first member of a cell matrix pair, a second member of a cell matrix pair, and a supporting cellular composition, and a pharmaceutically acceptable carrier, excipient, or diluent. In exemplary aspects, the pharmaceutical composition is sterile and suitable for administration to a subject, e.g., a human subject. In exemplary aspects, one or more components are encapsulated or conformally coated as described herein. In exemplary aspects, the therapeutic cells are encapsulated or conformally coated with a supporting cell composition or are encapsulated or conformally coated separately from the supporting cell composition, which may or may not be encapsulated or conformally coated. In exemplary aspects, the supporting cell composition comprises MSC or endothelial cells, and/or exosomes derived from MSC or endothelial cells.

In related embodiments, a composition prepared by the methods of preparing islet cells are also provided. In exemplary instances, the composition comprises pancreatic islet cells and MSC, endothelial cells, or exosomes derived therefrom. Optionally, the composition comprises about 100-500 pancreatic islet cells and about 10⁴ to about 10⁶ MSC or endothelial cells or about 10¹⁰ to about 10¹⁵ exosomes, per ml. The composition, in exemplary aspects, comprises pancreatic islet cells and the contents of exosomes derived from MSC or endothelial cells. The composition, in exemplary aspects, comprises pancreatic islet cells and one or more of: hepatocyte growth factor (HGF), thrombospondin-1 (TSP-1), a laminin, a collagen, insulin growth factor binding protein-1 (IGFBP-2), CD40, IGFBP-1, sTNFRII, CD40L, TNFα, cIAP-2, IGFBP-3, TNFβ, CytoC, IGFBP-4, TRAIL R1, TRAIL R2, TRAIL R3, bad, IGF-1 sR, TRAIL R4, HSP60, p27, Caspase 8, and IGF-2. The composition, in exemplary aspects, comprises pancreatic islet cells and one or more of: IGFB-1, IGFB-2, IGFB-3, IGFB-4, IGFB-6, IGF-1, IGF-1 SR, IGF-II, M-CSF, MCSF R, PDGF-AA, VEGF, IL-6, IL-8, Eotaxin, ICAM-1, IFNγ, CCL1, MCP-2, MIP-1α, RANTES, TNFα, or a combination thereof,

Methods of Use

Based on the information provided for the first time herein, it is contemplated that the methods of the present disclosures are useful for treatment of a disease or medical condition, in which a therapeutic agent, e.g., insulin, is deficient in the subject. Accordingly, the present disclosure provides a method of treating or preventing a disease or medical condition in a patient, wherein the disease or medical condition is a disease of medical condition in which a lack of insulin or insulin function is associated with the onset and/or progression of the disease of medical condition. The method comprises implanting the therapeutic cells, e.g., pancreatic islet cells, to the subject according to the methods of implanting therapeutic cells described herein, in an amount effective to treat or prevent the disease or medical condition.

In some embodiments, the disease or medical condition is Metabolic Syndrome. Metabolic Syndrome, also known as metabolic syndrome X, insulin resistance syndrome or Reaven's syndrome, is a disorder that affects over 50 million Americans. Metabolic Syndrome is typically characterized by a clustering of at least three or more of the following risk factors: (1) abdominal obesity (excessive fat tissue in and around the abdomen), (2) atherogenic dyslipidemia (blood fat disorders including high triglycerides, low HDL cholesterol and high LDL cholesterol that enhance the accumulation of plaque in the artery walls), (3) elevated blood pressure, (4) insulin resistance or glucose intolerance, (5) prothrombotic state (e.g., high fibrinogen or plasminogen activator inhibitor-1 in blood), and (6) pro-inflammatory state (e.g., elevated C-reactive protein in blood). Other risk factors may include aging, hormonal imbalance and genetic predisposition.

Metabolic Syndrome is associated with an increased the risk of coronary heart disease and other disorders related to the accumulation of vascular plaque, such as stroke and peripheral vascular disease, referred to as atherosclerotic cardiovascular disease (ASCVD). Patients with Metabolic Syndrome may progress from an insulin resistant state in its early stages to full blown type II diabetes with further increasing risk of ASCVD. Without intending to be bound by any particular theory, the relationship between insulin resistance, Metabolic Syndrome and vascular disease may involve one or more concurrent pathogenic mechanisms including impaired insulin-stimulated vasodilation, insulin resistance-associated reduction in NO availability due to enhanced oxidative stress, and abnormalities in adipocyte-derived hormones such as adiponectin (Lteif and Mather, Can. J. Cardiol. 20 (suppl. B):66B-76B (2004)).

According to the 2001 National Cholesterol Education Program Adult Treatment Panel (ATP III), any three of the following traits in the same individual meet the criteria for Metabolic Syndrome: (a) abdominal obesity (a waist circumference over 102 cm in men and over 88 cm in women); (b) serum triglycerides (150 mg/dl or above); (c) HDL cholesterol (40 mg/dl or lower in men and 50 mg/dl or lower in women); (d) blood pressure (130/85 or more); and (e) fasting blood glucose (110 mg/dl or above). According to the World Health Organization (WHO), an individual having high insulin levels (an elevated fasting blood glucose or an elevated post meal glucose alone) with at least two of the following criteria meets the criteria for Metabolic Syndrome: (a) abdominal obesity (waist to hip ratio of greater than 0.9, a body mass index of at least 30 kg/m2, or a waist measurement over 37 inches); (b) cholesterol panel showing a triglyceride level of at least 150 mg/dl or an HDL cholesterol lower than 35 mg/dl; (c) blood pressure of 140/90 or more, or on treatment for high blood pressure). (Mathur, Ruchi, “Metabolic Syndrome,” ed. Shiel, Jr., William C., MedicineNet.com, May 11, 2009).

For purposes herein, if an individual meets the criteria of either or both of the criteria set forth by the 2001 National Cholesterol Education Program Adult Treatment Panel or the WHO, that individual is considered as afflicted with Metabolic Syndrome.

Without being bound to any particular theory, the methods of implanting therapeutic cells described herein are useful for treating Metabolic Syndrome. Accordingly, the present disclosure provides a method of preventing or treating Metabolic Syndrome, or reducing one, two, three or more risk factors thereof, in a subject, comprising implanting therapeutic cells to the subject according to the methods described herein in an amount effective to prevent or treat Metabolic Syndrome, or the risk factor thereof.

In some embodiments, the method treats a hyperglycemic medical condition. In exemplary aspects, the hyperglycemic medical condition is diabetes, diabetes mellitus type I, diabetes mellitus type II, or gestational diabetes, either insulin-dependent or non-insulin-dependent. In some aspects, the method treats the hyperglycemic medical condition by reducing one or more complications of diabetes including nephropathy, retinopathy, and vascular disease.

The present disclosure provides a method of reducing an exogenous insulin requirement (EIR) of a subject with diabetes, comprising implanting cells in the subject according to any of the methods of implanting therapeutic cells described herein.

The present disclosure also provides a method of restoring euglycemia in a subject in need thereof, comprising implanting cells in the subject according to any of the methods of implanting therapeutic cells described herein.

The present disclosure further provides a method of inducing insulin-independence or reducing insulin resistance in a subject, comprising implanting cells in the subject according to any of the methods of implanting therapeutic cells described herein.

In some aspects, the disease or medical condition is obesity. In some aspects, the obesity is drug-induced obesity. In some aspects, the method treats obesity by preventing or reducing weight gain or increasing weight loss in the patient. In some aspects, the method treats obesity by reducing appetite, decreasing food intake, lowering the levels of fat in the patient, or decreasing the rate of movement of food through the gastrointestinal system.

Because obesity is associated with the onset or progression of other diseases, the methods of treating obesity are further useful in methods of reducing complications associated with obesity including vascular disease (coronary artery disease, stroke, peripheral vascular disease, ischemia reperfusion, etc.), hypertension, onset of diabetes type II, hyperlipidemia and musculoskeletal diseases. The present disclosures accordingly provides methods of treating or preventing these obesity-associated complications.

The present disclosure moreover provides a method of inducing weight loss in a subject, comprising implanting cells in the subject according to the method of any one of the preceding claims.

The following examples are given merely to illustrate the present invention and not in any way to limit its scope.

### EXAMPLES

### Example 1

This example describes the materials and methods used in the study in Example 2.

Animals

Studies involving animal subjects were performed under protocols approved and monitored by the University of Miami (UM) Institutional Animal Care and Use Committee. Animals were housed at the Division of Veterinary Resources. Lewis (MHC, rat haplotype: RT1^{I}) and Wistar Furth (WF) (RT1^{U}) rats (http://www.harlan.com) were used as islet donors (>250 g males) and Lewis rats as recipients (170-200 g females). For selected experiments, rats with an indwelling jugular vein catheter (JVC) were purchased. Rodents were housed in microisolated cages with free access to autoclaved water and food. Both donor and recipient cynomolgus monkeys (7.92 and 3.58 years old, respectively) were obtained from the Mannheimer Foundation (Homestead, Fla.) and were specific pathogen free. Pair-housed monkeys were supplied with water ad libitum and fed twice daily (24). UM holds Animal Welfare Assurance A-3224-01, effective 12 Apr. 2002, with the Office of Laboratory Animal Welfare, National Institutes of Health, and is accredited by the Association for Assessment and Accreditation of Laboratory Animal Care.

Diabetes Induction and Metabolic Monitoring

Diabetes was induced by administration of streptozotocin at 60 mg/kg i.p. in rats (2 injections 2-3 days apart; Sigma-Aldrich) (25) and 100 mg/kg i.v. in NHP (Tevapharm.com) (7,22).

Rodents with nonfasting glycemic values >300 mg/dL⁻¹ on whole blood samples obtained from tail pricking (OneTouch Ultra glucometers [http://www.lifescan.com]) were used as recipients. Graft function was defined as nonfasting glycemia <200 mg/dL. At selected time points after transplantation, a glucose tolerance test was performed in rodents to evaluate graft potency (25). After overnight fasting, an oral (oral glucose tolerance test [OGTT]; 2.5 g/kg) or intravenous (intravenous glucose tolerance test [IVGTT]; 0.5 g/kg) glucose bolus was administered, and glycemic values were monitored with portable glucometers. The area under the curve (AUC) of glucose was calculated as previously described (26). In the case of allogeneic islet transplants, return to nonfasting hyperglycemic state was considered a sign of graft rejection.

In NHP, diabetes was defined as fasting C-peptide levels <0.2 ng/mL and a negative response (stimulated C-peptide <0.3 ng/mL) to a glucagon challenge performed 4 weeks after streptozotocin treatment (7,22). Heel-stick glycemic values were monitored two to three times daily (OneTouch Ultra). Subcutaneous insulin (Humulin R [https://www.lilly.com] or Humulin R plus Lantus [http://www.sanofi.us]) was administered based on an individualized sliding scale as needed, aiming for fasting blood glucose (FBG) and postprandial plasma blood glucose (PBG) levels of 150-250 mg/dL⁻¹ poststreptozotocin and prior to transplantation. Plasma C-peptide levels were assessed by electrochemiluminescence immunoassay using a Cobas analyzer (https://usdiagnostics.roche.com).

Plasma Collection

Blood obtained from venipuncture was collected into microcentrifuge tubes containing 3.2% sodium citrate. Plasma was obtained after centrifugation at 1,455 g for 10 min at room temperature, and aliquots were stored at −80° C. and thawed before use.

Islet Isolation and Graft Preparation

Islets were obtained by enzymatic digestion, followed by purification on density gradients using protocols standardized at the Diabetes Research Institute (DRI) for rats (DRI Translational Core) (27), NHP (22), and humans (DRI cGMP Human Cell Processing Facility) (1,28-30).

For evaluation of the effect of the degree of islet preparation purity on intraomental engraftment and function, Lewis rat islets were isolated and purified using a standard technique (27), yielding >95% purity (pure fraction) as assessed by dithizone staining (Sigma-Aldrich) (1,2). The pancreatic slurry containing exocrine tissue clusters (lowest purity fraction) after islet purification was maintained in culture and counted with the algorithm used to determine islet equivalents (IEQ) (2). Before transplantation, an aliquot of the final pure islet product was mixed with the exocrine pancreatic tissue to obtain a 30% pure islet cell product (3:7 v/v, islet vs. exocrine tissue).

Islet Transplantation

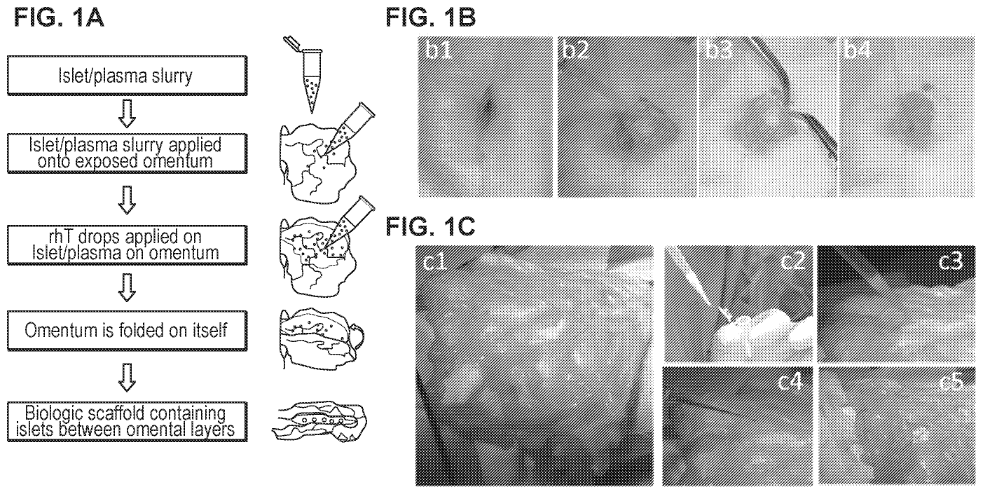

Under general anesthesia, a substernal midline minilaparotomy allowed exteriorizing the omentum that was spread flat over a sterile field (FIG. 1). We previously reported a similar intraomental flap transplant procedure in NHP (22).

The rhT (Recothrom, National Drug Code no. 28400 [http://www.zymogenetics.com]) was reconstituted with the 0.9% NaCl included in the kit for rodents or with Dulbecco PBS with Ca²⁺/Mg²⁺ (GIBCO [http://www.thermofisher.com]) in the NHP experiment. Final aliquots of rhT (1,000 IU/mL) were stored at −20° C.

The transplantation procedure with the in situgeneration of the islet-containing biologic scaffold is summarized in FIG. 1. For rodent experiments, islet aliquots were centrifuged (1 min, 200 g), supernatant was discarded, and islets were resuspended in syngeneic (autologous) plasma. After another centrifugation, most of the excess plasma was removed and the slurry of islets/plasma collected with a precision syringe (hamiltoncompany.com). Similarly, in the case of the NHP, islets were collected into a microcentrifuge tube, quick spun and washed twice in donor plasma (obtained on day −1, stored at 4° C.), and transported to the operating room. Plasma excess was removed before collection of islets/plasma with use of a micropipette (P1000). In both species, the islets/plasma slurry was gently distributed onto the surface of the omentum (FIG. 1A, b2, and c3) and then rhT gently dripped onto the graft (FIG. 1A, c4), resulting in immediate gelling and adherence of the islets to the omental surface. The omentum was gently folded on itself to increase contact with and containment of the graft (FIG. 1A, b3, b4, and c5). In the NHP, nonresorbable sutures were placed on the omentum outside the graft area as reference for the time of retrieval (FIG. 1, c5). In rodent experiments, nothing, sutures, or a plasma:rhT mix (10:1 v/v) was used on the folded omentum. After repositioning the omentum into the peritoneal cavity, abdominal wall muscle and skin were sutured.

To avoid the potential confounding effect of variability in islet preparations (31), for rat experiments aimed at comparison of transplant sites or purity, equal numbers of syngeneic islets, from large batch isolations, were implanted in parallel into diabetic recipients in a biologic scaffold or into the liver (25). In selected rats, survival surgery to remove graft-bearing omentum after long-term follow-up was performed to confirm prompt return to hyperglycemia, thereby ruling out residual function of native endocrine pancreas.

Immunosuppression

Clinically relevant immunosuppressive agents were used in both rat and NHP allogeneic transplant models (32-35). Rats received induction treatment with antilymphocyte serum (0.5 mL i.p. on day −3 [http://www.accurate.com.gr]) and mycophenolic acid (20 mg/kg/day starting on the day of transplant for 2 weeks then tapered by one-quarter of the dose every 2 days until day 20; Myfortic [https://www.novartis.com]) (36) combined with cytotoxic T-lymphocyte-associated protein-4 (CD152)-immunoglobulin fusion protein (CTLA4Ig) (10 mg/kg i.p. on days 0, 2, 4, 6, 8, and 10 and weekly thereafter; abatacept [Orencia] [http://www.bms.com]), adapted from Safley et al. (35). The NHP received anti-thymocyte (rabbit) globulin (10 mg/kg i.v. on days −1, 0, 2, and 4 from transplant; thymoglobulin; Sanofi), CTLA4Ig (20 mg/kg i.v. on days 0, 4, 14, 28, 56, and 75 and monthly thereafter at 10 mg/kg; belatacept [Nulojix] [http://www.bms.com]), and sirolimus (daily from day 2, aiming at trough levels of 8-12 ng/mL; rapamycin [http://www.lclabs.com]).

Biomarkers

Blood samples were collected from indwelling JVC at baseline and after selected time points posttransplant. Insulin, C-peptide, leptin, interleukin (IL)-6, and chemokine (C-C motif) ligand 2 (CCL2) (formerly MCP-1) were measured using commercial multiplex kits (http://www.emdmillipore.com) and analyzed on a Luminex system. Levels of the rat acute-phase proteins α2-macroglobulin and haptoglobin were measured using specific ELISA kits (http://www.lifediagnostics.com) on a kinetic microplate reader (SoftMaxPro version 5; https://www.moleculardevices.com).

Histopathology

Tissue sections (4 μm thick) were stained with hematoxylin-eosin for morphologic assessment of the grafts. Masson trichrome staining (Chromaview, Richard-Allan Scientific [https://vwr.com]) was performed on selected grafts to reveal collagen (blue stain) or muscle fibers and cytoplasm (red stain) (25). Immunofluorescence was performed using specific antibodies to detect insulin (1:100; guinea pig anti-insulin [http://www.dako.com]), glucagon (GCG) (1:100; rabbit anti-glucagon [http://www.biogenex.com]), endothelium (1:20; rabbit anti-CD31 [http://www.abcam.com] or 1:50; von Willebrand factor [vWF], rabbit anti-vWf [http://www.emdmillipore.com]), smooth muscle actin (SMA) (1:50; rabbit anti-SMA [http://www.abcam.com]), and T cells (1:100; CD3, rabbit polyclonal anti-human [http://www.cellmarque.com]). Secondary antibodies used were goat anti-guinea pig Alexa Fluor 568 (1:200) and goat anti-rabbit Alexa Fluor 488 (1:200; Invitrogen [http://www.thermofisher.com]). Digital images were acquired using an SP5 inverted confocal microscope (http://www.leica.com) at the DRI Imaging Core Facility.

Scanning Electron Microscopy

Human plasma was obtained from volunteers who gave consent (IRB20091138). Human islets and plasma/thrombin clots with or without human islets were fixed in 2% glutaraldehyde in PBS (0.137 mol/L NaCl, 0.01 mol/L Na₂HPO₄, and 0.0027 mol/L KCl, pH 7.4) for >3 h and stored in fixative at 4° C. After three washes, samples were postfixed with 1% osmium tetroxide in PBS, dehydrated in ethanol, dried with hexamethyldisilazane, dispersed in plastic weigh boats, and outgassed overnight. Preparations were adhered by gentle tapping with an aluminum stub, covered with a carbon adhesive tab, and then coated with a 20-nm-thick layer of palladium in a plasma sputter coater and imaged at the UM Center for Advanced Microscopy using a field emission scanning electron microscope (FEI XL-30).

Statistical Analysis

Data were analyzed using Excel (https://www.microsoft.com), SigmaPlot (http://www.sigmaplot.com), and Prism6 (http://www.graphpad.com). Shapiro-Wilk test was used to assess parametric data distribution. An unpaired t test was performed to compare experimental groups. Values shown are mean±SEM, except where indicated. P values <0.05 were considered statistically significant.

### Example 2

This example demonstrates a method of implanting cells into non-human subjects.

Transplantation of pancreatic islets is a therapeutic option to preserve or restore β-cell function. Our study was aimed at developing a clinically applicable protocol for extrahepatic transplantation of pancreatic islets. The potency of islets implanted onto the omentum, using an in situ-generated adherent, resorbable plasma-thrombin biologic scaffold, was evaluated in diabetic rat and nonhuman primate (NHP) models. Intraomental islet engraftment in the biologic scaffold was confirmed by achievement of improved metabolic function and preservation of islet cytoarchitecture, with reconstitution of rich intrainsular vascular networks in both species. Long-term nonfasting normoglycemia and adequate glucose clearance (tolerance tests) were achieved in both intrahepatic and intraomental sites in rats. Intraomental graft recipients displayed lower levels of serum biomarkers of islet distress (e.g., acute serum insulin) and inflammation (e.g., leptin and α2-macroglobulin). Importantly, low-purity (30:70% endocrine:exocrine) syngeneic rat islet preparations displayed function equivalent to that of pure (>95% endocrine) preparations after intraomental biologic scaffold implantation. Moreover, the biologic scaffold sustained allogeneic islet engraftment in immunosuppressed recipients.

Ultramicroscopic Structure of the Biologic Scaffold

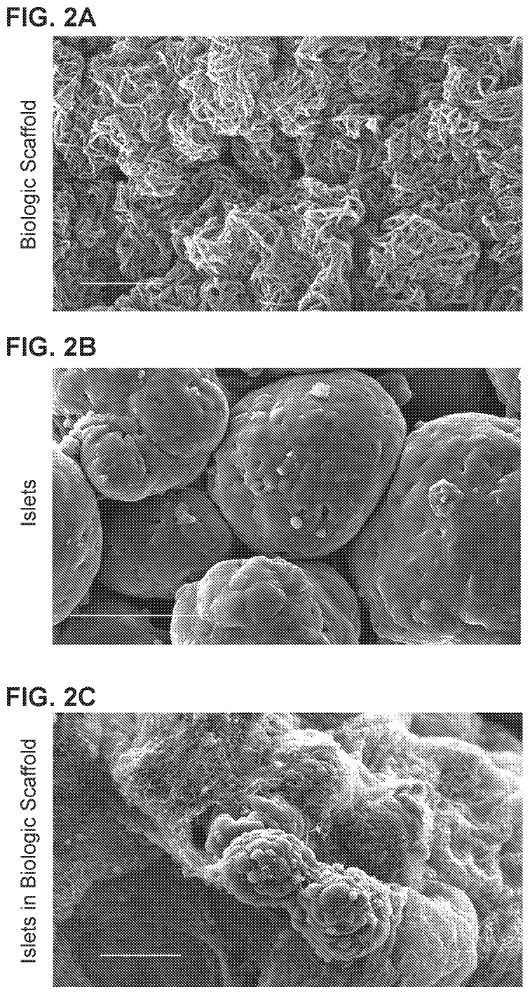

Scanning electron microscopy revealed the intricate net of fibrin fibers obtained through the reaction triggered by rhT in human plasma (FIG. 2A). Human islet cell surface appeared smooth by ultramicroscopy (FIG. 2B). The clot induced by combining human plasma with rhT and human islets in vitro resulted in the development of an orthomorphic three-dimensional fibrin matrix trapping islet structures within the newly formed biologic scaffold (FIG. 2C). We reasoned that the induction of the plasma/thrombin reaction to create a microscopic, adherent fibrin scaffold around the implanted islets would be useful to promote islet graft adhesion on the surface of the omentum, preventing islet pelleting, and therefore aiding engraftment, neovascularization, and islet survival.

Intraomental Islets Transplanted into Biologic Scaffolds Restore Normoglycemia in Diabetic Rats

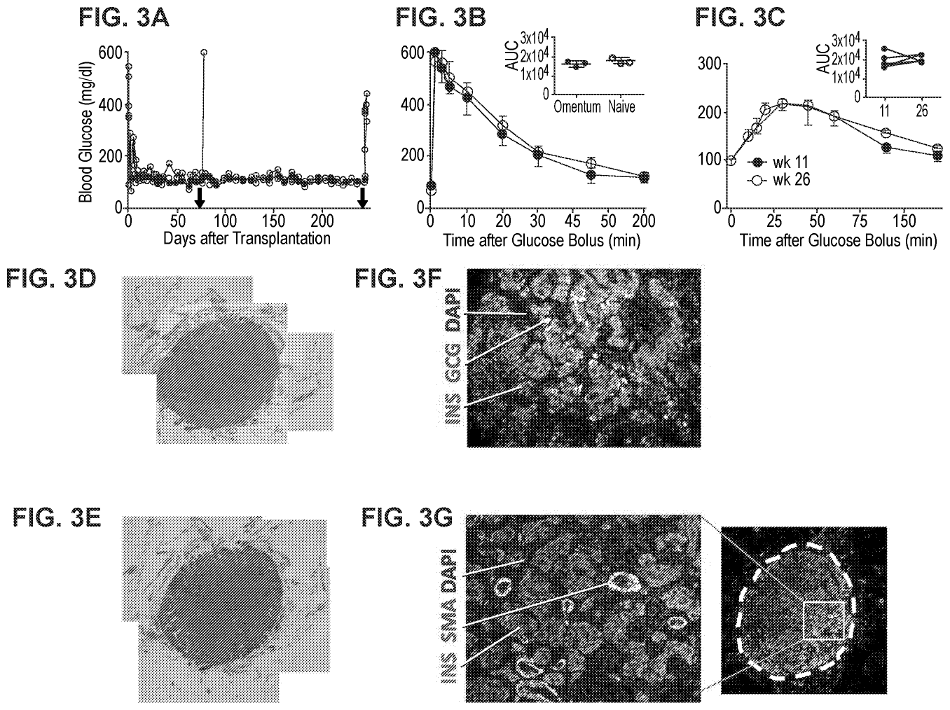

A syngeneic rat islet transplant model was used. It was previously demonstrated that streptozotocin diabetic recipients of 3,000 IEQ experienced reversal of diabetes after either intra- or extrahepatic transplantation (25). After intraomental transplantation of 17,338±881 IEQ/kg, all animals (n=7; 173.4±91 g body wt) achieved normoglycemia within 2 days and maintained euglycemia during the follow-up period (FIG. 3A), even >200 days. Two animals underwent removal of the graft-bearing omentum on day 76 posttransplantation: one died after surgery and the other promptly become hyperglycemic (FIG. 3A). The rest of the recipients were followed for >200 days posttransplantation. IVGTT performed in selected animals 2 months after islet transplantation showed that transplanted islets cleared glucose within 75 min after receiving a glucose bolus in a fashion comparable with that of naïve animals (n=3/group; AUC 17,851 810 mg×min×dL⁻¹ in naïve rats vs. 16,276 857 mg×min×dL⁻¹ in intraomental islet biologic scaffold recipients) (FIG. 3B). Furthermore, OGTT performed on transplanted animals at 11 and 26 weeks postimplantation confirmed comparable glucose clearance at both time points (n=5; AUC 19,542 1,735 mg×min×dL⁻¹ at 11 weeks and 20,735 785 mg×min×dL⁻¹ at 26 weeks) (FIG. 3C). Histopathology of explanted grafts showed well-preserved islet cytoarchitecture (FIG. 3D-G), strong insulin immunostaining, and abundant intragraft vascularization (e.g., SMA); all features were compatible with adequate engraftment and corroborated the in vivo functional data.

Comparable Function of Intrahepatic and Intraomental Islets Transplanted into Biologic Scaffolds

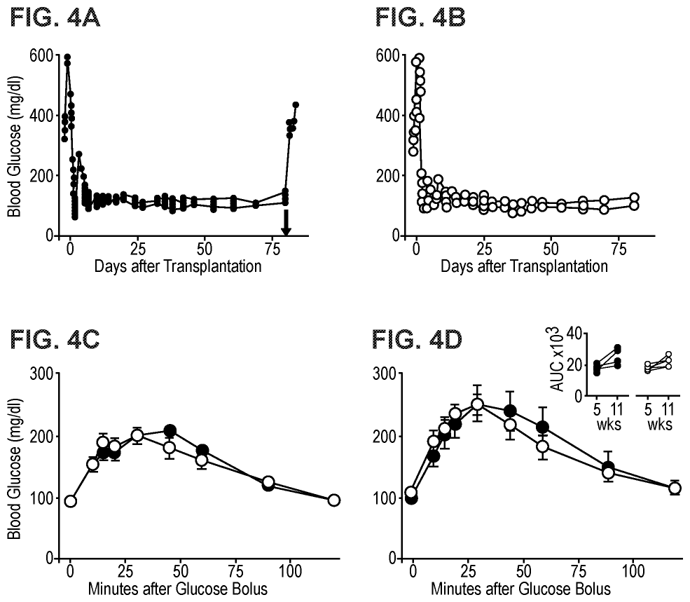

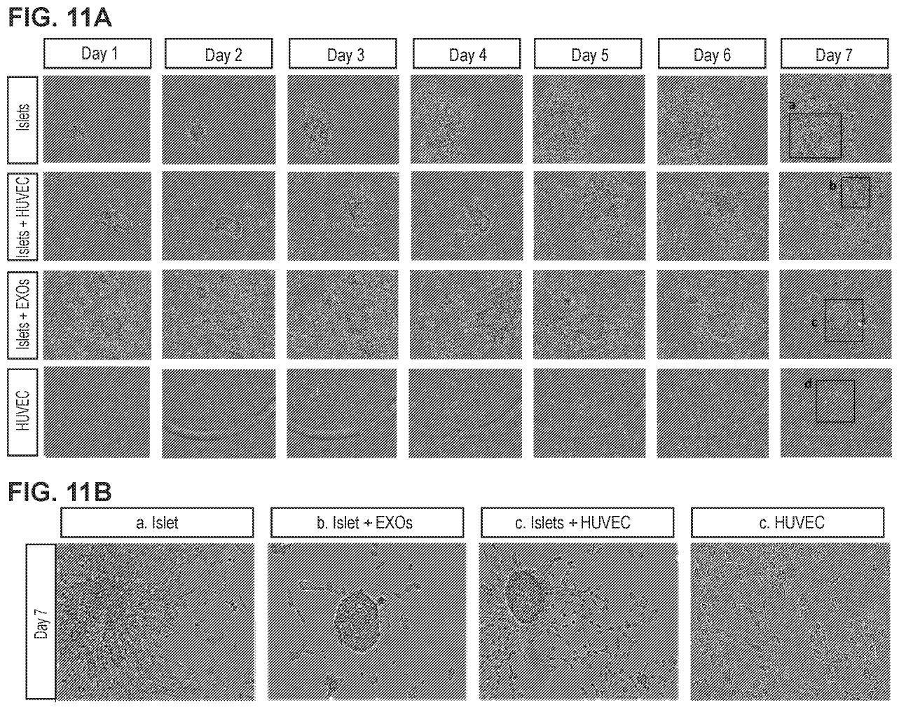

The performance of syngeneic islets implanted within the intraomental biologic scaffold was compared to that of intrahepatic grafts. Aliquots of 1,300 IEQ (˜8,200 IEQ/kg body wt [a “clinically relevant” mass]) from the same batch of islets were transplanted in parallel either within an intraomental biologic scaffold (n=7; 160.3±6.4 g body wt [8,122±334 IEQ/kg]) (FIG. 4A) or in the intrahepatic site (n=5; 155.4 7.3 g body wt [8,380 396 IEQ/kg]) (FIG. 4B). All recipients in both groups achieved euglycemia within 1 week and maintained good metabolic control during the 82-day (˜12 weeks) follow-up period. Omental graft removal resulted in a prompt return to hyperglycemia (n=4) (FIG. 4A). At 5 (FIG. 4C) and 11 weeks posttransplant (FIG. 4D), OGTT showed comparable metabolic function in both transplant sites (AUC at 5 weeks, 18,393±571 and 18,036±598.5 mg×min×dL⁻¹, and AUC at 11 weeks, 21,987±2,580 and 21,149 1,456 mg×min×dL⁻¹, for intraomental biologic scaffold recipients or intrahepatic islet recipients, respectively) (FIGS. 4C and D).

Lower Levels of Stress-Related Biomarkers in Recipients of Intraomental Islets within a Biologic Scaffold

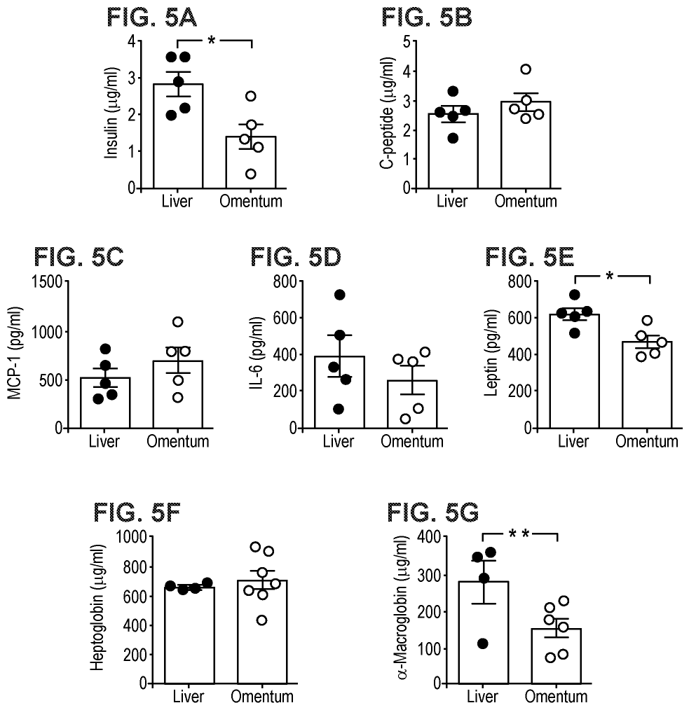

Selected biomarkers associated with islet distress and inflammation elicited by the transplantation procedure were evaluated. Blood samples were collected from JVC at different time points after transplantation. A spike in insulin and C-peptide levels, likely a result of insulin dumping from distressed islet cells (5,37,38), was observed 1 h posttransplant in both experimental groups. The insulin peak was significantly higher in the intrahepatic compared with the intraomental group (2.841±0.338 vs. 1.405±0.352 μg/mL, respectively; P=0.018) (FIG. 5A), with comparable levels in both groups at subsequent time points (data not shown). No statistically significant differences were observed in C-peptide levels (2.565±0.25 vs. 2.941±0.303 μg/mL, respectively) (FIG. 5B).

Intraomental Biologic Scaffold Provides Adequate Engraftment of Low-Purity Islet Preparations

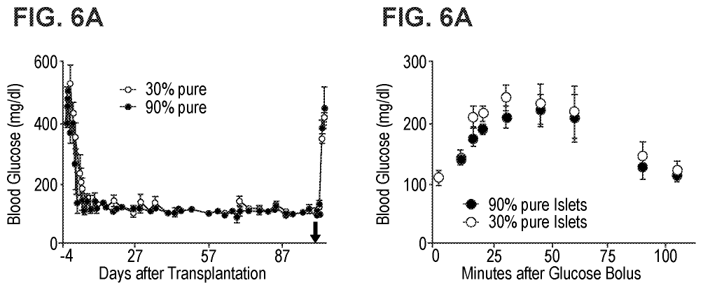

Clinical human islet preparations usually contain different degrees of impurities (e.g., exocrine tissue) that increase the final volume of transplanted tissue. We evaluated whether our intraomental biologic scaffold would be adequate for the implantation of clinically relevant, low-purity islet preparations. Implantation of 2,000 IEQ from either >95% pure (n=3; 167.3±1.5 g body wt [11,853±109 IEQ/kg]) or 30% pure (n=3; 170.3±10.5 g body wt [11,771±725 IEQ/kg]) syngeneic islet preparations led to rapid diabetes reversal (within 5 days) in all recipient rats (FIG. 6A). All animals maintained stable normoglycemia throughout the follow-up period and displayed comparable glucose clearance during OGTT (AUC: 17,776±1,687 and 19,734±1,997 mg×min×dL⁻¹ for recipients of 90 and 30% pure preparations, respectively) (FIG. 6B). Surgical removal of the graft-bearing omentum was performed >100 days after transplantation. One of the recipients of 30% pure islets died after surgery, while all other animals in both groups showed prompt return to hyperglycemia, confirming the graft-dependent normoglycemia.

Intraomental Biologic Scaffold Provides Adequate Engraftment of Allogeneic Islets in Immunosuppressed Recipients

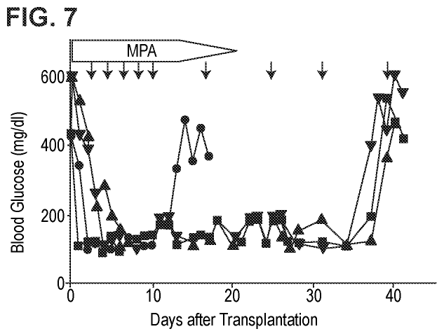

Suitability of the intraomental biologic scaffold to support islet engraftment under clinically relevant systemic immunosuppressive treatment (32-35) was evaluated in a fully MHC-mismatched allogeneic rat transplant combination. All four diabetic Lewis rat (RT1ⁱ) recipients of 3,000 IEQ WF islets (RT1^{u}) achieved normoglycemia within 5 days and sustained graft function for up to 5 weeks posttransplantation under the transient systemic immunosuppression protocol used, when graft rejection coincided with return to a hyperglycemic state (FIG. 7).

Intraomental Islet Transplant in a Biologic Scaffold Engrafts in a Preclinical Model

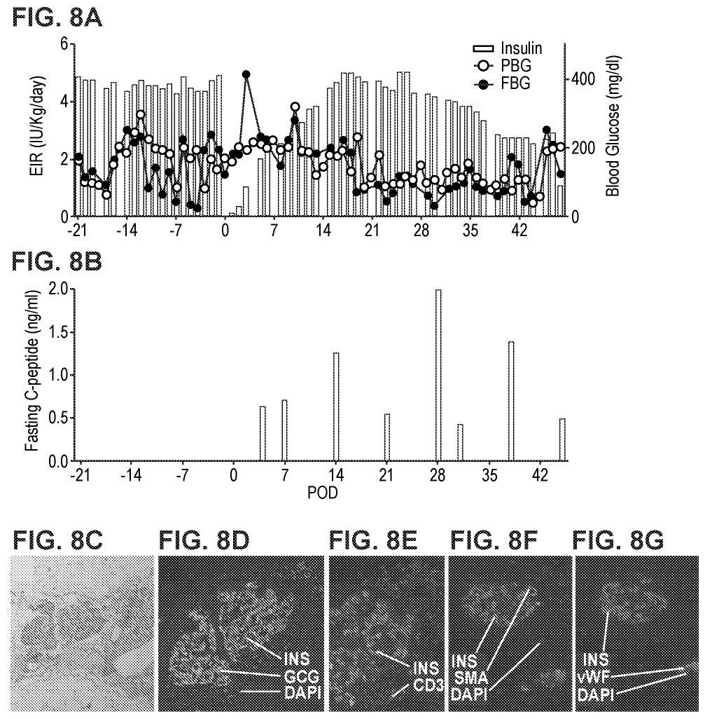

The effect of the biologic scaffold was examined in a clinically relevant preclinical model of allogeneic islet transplantation in a diabetic cynomolgus monkey. Before transplantation, the animal required ˜4-5 IU/kg/day exogenous insulin with plasma C-peptide <0.05 ng/mL (FIG. 8). An islet mass of ˜48,700 IEQ (˜150 μL total islet graft volume) equivalent to 9,347 IEQ/kg was implanted. The transplant procedure and postoperative clinical outcome were uneventful, with standard recovery from surgery. After the first few weeks posttransplant, improvement of FBG and PBG was observed, requiring progressive reduction of exogenous insulin (FIG. 8A). Positive fasting C-peptide levels (FIG. 8B) were observed immediately posttransplant and throughout follow-up. The animal subsequently expired on postoperative day (POD) 49 owing to technical complications unrelated to the engraftment site. Histopathologic assessment of the explanted graft demonstrated well-preserved islet morphology (FIG. 8C) with immunoreactivity for the endocrine markers insulin and glucagon (FIG. 8D), some degree of peri-insular lymphocyte infiltrate (CD3) (FIG. 8E), and abundant intra- and extrainsular vascular structures (SMA [FIG. 8F] and vWF [FIG. 8G]).

### Example 3

This example demonstrates an exemplary method of implanting cells into a human subject.

Islet transplantation can restore euglycemia and eliminate severe hypoglycemia in patients with type 1 diabetes.¹ When islet transplantation is performed with intra-hepatic placement, limitations include a limited transplant tissue volume, bleeding with placement, exposure to high immunosuppressive drug levels after transplant and the triggering of an immediate blood-mediated inflammatory response.² The omentum offers a dense vascularized surface for islet implantation, drains into the portal system and is easily accessible.^{3,4}

The case of a 43-year-old woman with a 25-year history of type 1 diabetes (weight 53.4 Kg; BMI 21.5 Kg/m²; insulin daily dose 32.91±1.28 units/day) that was complicated by hypoglycemia unawareness and episodes of severe hypoglycemia is presented here. The stimulated C-peptide level was <0.3 ng/mL (<0.1 nmol/L) and HbA1c was 6.8%. The patient underwent islet transplantation onto the omentum (NCT02213003); 602,395 islet equivalents (1 islet equivalent=150 μm islet diameter) from a single deceased-donor (6.5 mL total packed tissue volume) were combined with autologous-plasma (1:2 ratio) and laparoscopically layered onto the omentum. Recombinant thrombin (20 mL; Recothrom®, 1000 units/mL) was layered over the islets. Unlike the methods described in Example 1 and 2, another layer of autologous plasma was layered after thrombin was layered over the islets to generate a degradable biologic scaffold. Induction comprised anti-thymocyte globulin and etanercept, and maintenance immunosuppression consisted of mycophenolate sodium and tacrolimus. Tacrolimus was switched to sirolimus 8 months after transplant due to hair loss. No surgical complications were observed.

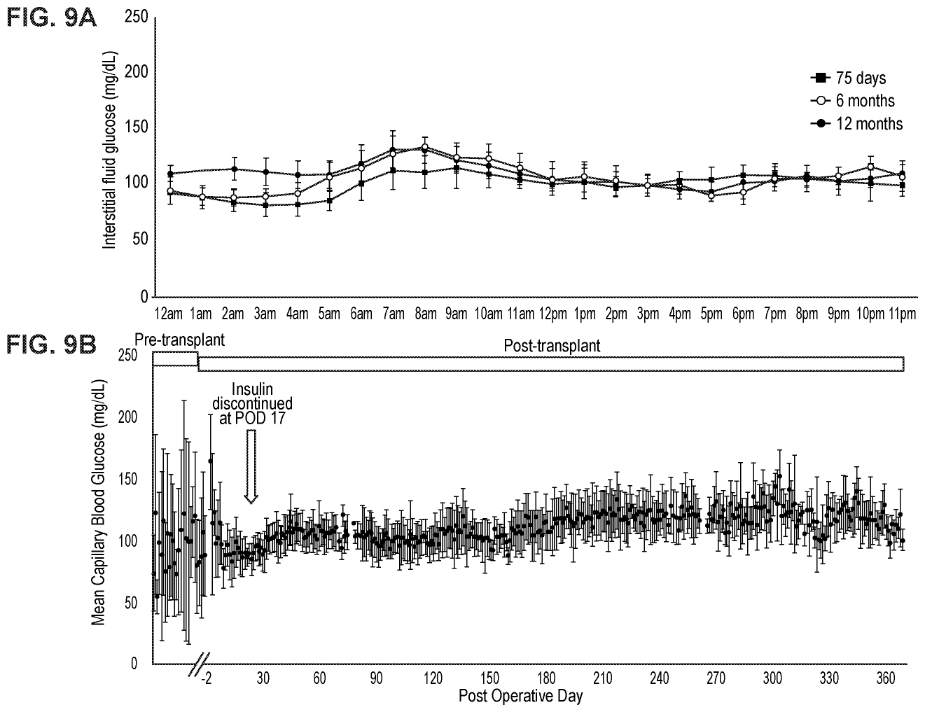

Insulin was discontinued 17 days following transplant. Capillary blood glucose, continuous glucose monitoring, mixed meal tolerance test, HOMA indexes and BETA scores⁵ are shown in FIG. 9 and Table 1.

TABLE 1

| | Pre- | | | |
|---|---|---|---|---|
| | transplant | 75 days | 6 months | 12 months |
| MMTT Variables | | | | |
| Fasting glucose | 105 | 107 | 91 | 120 |
| (mg/dL) | | | | |
| 90-min glucose | 298 | 175 | 165 | 266 |
| (mg/dL) | | | | |
| Peak glucose | 298 | — | 181 | 277 |
| (mg/dL) | | | | |
| Fasting C-peptide | 0.05 | 0.79 | 0.56 | 0.43 |
| (ng/mL) | | | | |
| 90-min C-peptide | 0.15 | 2.79 | 3.27 | 1.22 |
| (ng/mL) | | | | |
| Peak C-peptide | 0.18 | — | 3.32 | 1.79 |
| (ng/mL) | | | | |
| HOMA indices | | | | |
| HOMA2-% B | | 47.0 | 52.7 | 24.8 |
| HOMA2-% S | | 165.5 | 238.9 | 296.4 |
| HOMA2-IR | | 0.6 | 0.4 | 0.3 |
| BETA scores | | | | |
| B-score | | 7 | 8 | 7 |
| BETA-2 score | | 16 | 15 | 10 |

At 12 months, 90-minute glucose was 266 mg/dL⁻¹ (14.6 mmol/L) decreasing to 130 mg/dL⁻¹ (7.1 mmol/L) at 300-minutes. Continuous glucose monitoring 7-day mean glucose was 109±15 mg/dl (6.0±0.8 mmol/L; n=1876), HbA1c 6.0%, β-score 7 and BETA-2 score 10. Insulin secretion declined over time, and glucose levels increased, though these remained within the sub-diabetic range. The patient presently exercises regularly and follows a low-carbohydrate diet, which likely contribute to her glycemic stability.

In this patient islet transplantation onto the omentum restored euglycemia and insulin independence. A functional decline was observed at 12 months with an increase in insulin sensitivity, which we think may be due to conversion of tacrolimus to sirolimus. The patient currently has stable glycemic control without exogenous insulin and without episodes of hypoglycemia.

Supplementary Information Materials and Methods

Clinical History at Onset of Type 1 Diabetes

The patient reported here received a diagnosis of type 1 diabetes at 17 years of age, after she presented with polydipsia, polyuria, polyphagia and an unintended weight loss of approximately 10 lbs over the previous month. She recalled that she had had a glucose level in the 800 mg/dL⁻¹ range at disease onset but could not remember the HbA1c level or confirm a diagnosis of diabetic ketoacidosis. Medical records related to her initial diagnosis are not available, as the patient was living overseas at the time. She was immediately started on insulin therapy.

Pre-Transplant Evaluation

At the time of screening evaluation for islet transplantation, insulin requirements obtained from a 28-day glucose-insulin log were 33.30±1.76 units/day. The patient had a Clarke score of 6, consistent with hypoglycemia unawareness.¹ She was experiencing severe hypoglycemia (i.e., requiring assistance) at least once per month during the year before transplantation and had experienced three episodes associated with loss of consciousness requiring glucagon administration.

Pre-transplant evaluation was notable for a fasting C-peptide <0.1 ng/mL (<0.03 nmol/L) and 90-minute C-peptide (following a 2 hr mixed meal tolerance test) of 0.15 ng/mL (0.05 nmol/L) with a corresponding glucose of 298 mg/dL⁻¹ (16.39 mmol/L). We assessed the presence of diabetes-associated autoantibodies to glutamic acid decarboxylase (GAD65), insulinoma antigen-2 (IA-2), Zinc transporter protein 8 (ZnT8) and insulin. With the exception of the insulin antibody assay, all autoantibody levels are expressed as an index calculated from the counts per minute of the test sample and the positive and negative control samples. The upper limits of normal were calculated using ROC (Receiver Operating Curves). The patient's results were positive for IA-2 (index 23.3 [normal cut-off 6.4]) and ZnT8 (index 7.9 [normal cut off 3.35]) autoantibodies but negative for GAD65 and insulin autoantibodies. On a 5 month pre-transplant follow-up visit, insulin requirements from 28-day glucose-insulin log were 32.91±1.28 units/day.

Weight Loss after Islet Transplantation

Following islet transplantation, a gradual weight loss of 8.2 Kg was observed by 6 months post-transplant. Her weight stabilized and plateaued at 45.5 Kg after a nutritional evaluation and resumption of a balanced diet. Weight loss post islet transplantation is not unexpected and has been previously reported.² Possible contributing factors include optimization of insulin requirements (i.e., elimination of exogenous insulin administration and physiologic endogenous insulin delivery) and a reduction in carbohydrate intake, in part due to the resolution of hypoglycemia.

Homeostatic Model Assessment (HOMA) Indexes

HOMA indexes³ were calculated to monitor changes in insulin sensitivity and beta cell function longitudinally using the HOMA2 calculator (version 2.2.3© Diabetes Trials Unit, University of Oxford) available online at http://www.dtu,ox.ac.uk/homacalculator.

HOMA2-IR (insulin resistance) declined overtime. HOMA2-% B (13-cell function) improved by 6 months in line with metabolic responses observed during MMTT. A decline in HOMA2-% B was observed at 12 months, which was accompanied by a progressive increase in HOMA-2% S (insulin sensitivity).

BETA Scores

The validated 13-score⁴ and BETA-2 score⁵ were utilized as composite measures of beta cell function following islet transplantation.

At 6 months, she had a β-score of 8, which indicated overall excellent graft function, while the BETA-2 score of 15, associated with insulin independence, provided further information regarding glycemic control as this value is also associated with glucose intolerance and the patient had a 90-minute glucose of 165 mg/dL⁻¹ (FIG. 9., Panel C).

At 12 months, the β-score decreased to 7, a value still associated with insulin independence and overall good graft function. Notably, the BETA-2 score markedly decreased to 10, a value generally associated with loss of insulin independence. Although the patient had decreased insulin secretory responses to a mixed meal tolerance test, her overall glycemic control evaluated by 7-day continuous glucose monitoring was 109±15 mg/dL⁻¹ (range 73-151 mg/dL, n=1876) not necessitating reintroduction of insulin therapy. The patient has continued to exercise regularly doing mostly aerobic exercises and follows a healthy low carbohydrate diet. We speculate that these factors likely contribute to her stable glycemic control.

### Example 4

This example demonstrates an exemplary method of preparing pancreatic islets.

Destruction of insulin producing cells in the Islets of Langerhans occurs during the progression of Type 1 Diabetes Mellitus (T1DM), leading to loss of glucose homeostasis. Transplantation of human islets to cure T1DM is the only cell source currently used for cell-based therapies. Islet transplantation in recipients with T1DM improves blood glucose control, reduces or eliminates the need for exogenous insulin injections to control hyperglycemia, prevents severe hypoglycemic episodes and maintains near normal HbA1c levels. However, the efficacy of transplantation has been limited due to loss of islet viability and function during pre-transplantation or to islet rejection, or loss of graft function post-transplantation. Enhancement of islet viability prior to transplantation and improvement in long term function may improve clinical outcome. The aim of this study was to elucidate the positive effect of endothelial cells and endothelial-derived exosomes on pancreatic islet functionality and viability in vitro. Co-culture of islets with endothelial cells or endothelial-derived exosomes was able to maintain islet integrity, morphology and prolong the functionality of pancreatic islets. These results demonstrate that endothelial-derived exosomes can be used as a cell-free therapy for the optimization of pre-transplant culture of human islets, potentially to prolong human pancreatic islets functionality and clinical outcomes.

Transplantation, either of the whole pancreas or of purified islet cells is considered a therapeutic option to restore insulin secretion in patients with type 1 diabetes (T1D) 1. Islet transplantation has been shown to improve blood glucose control, reducing or eliminating the need for insulin injections to control diabetes, while preventing severe hypoglycemic episodes and maintaining near normal HbA1c levels in recipients with T1D². However the efficacy of islet transplantation has been limited, and thus there is a significant need for improvement of the success rate in post-transplant outcomes of islet transplants 4. One of the limitations includes the loss of human pancreatic islet viability and function during the 2-3 day in vitro culture pre-transplantation 5. Since in vitro culture is required to perform quality controls and ensure that islets meet product release criteria pre-transplant, it is important to optimize islet survival and overall quality during culture.

In order to improve islet transplantation for the treatment of diabetes, islets can be co-cultured with different cell types such as mesenchymal stem cells or endothelial cells₇₁₂. Indeed, culturing islets with endothelial cells improves the survival and function of pancreatic islets^{7,13} and may improve the success of the transplantation. Also certain factors secreted by endothelial cells, such as VEGF or human interleukin-1 receptor antagonist (hlL-1Ra), may be able to prolong islets survival^{3,14,15} Similarly, other paracrine factors released by endothelial cells such as microvesicles appear able to improve β-cell function^{16,17}. The aim of the present study was to investigate further the capability of endothelial cells and, in particular, endothelial-derived exosomes to preserve and improve survival and function of human pancreatic islets in culture pre-transplant.

Results

Endothelial Cells and Endothelial Cell-Derived Exosomes Characterization

Almost all cells, including HUVECs¹⁸, release different types of membrane microvesicles and nanovesicles, which have a variety of important physiological effects. Microvesicles differ from nanovesicles mainly by their size and mechanism of generation. Microvesicles are released from the plasma membrane by shedding or budding, are usually larger than 0.2 μm in size and have been referred to as microparticles or ectosomes. By contrast, nanovesicles including exosomes are between 30-100 nm in diameter, characterized by an endocytic origin and formed by the reverse budding of the peripheral membrane of multi-vesicular bodies (MVBs) or late endosomes. The protein content of different types of extracellular vesicles reflects, in general, that of the parent cells. EVs also are enriched in certain molecules including proteins and RNAs. Here we refer to the nanovesicles produced by HUVECs as exosomes due to their size and protein content (see below).

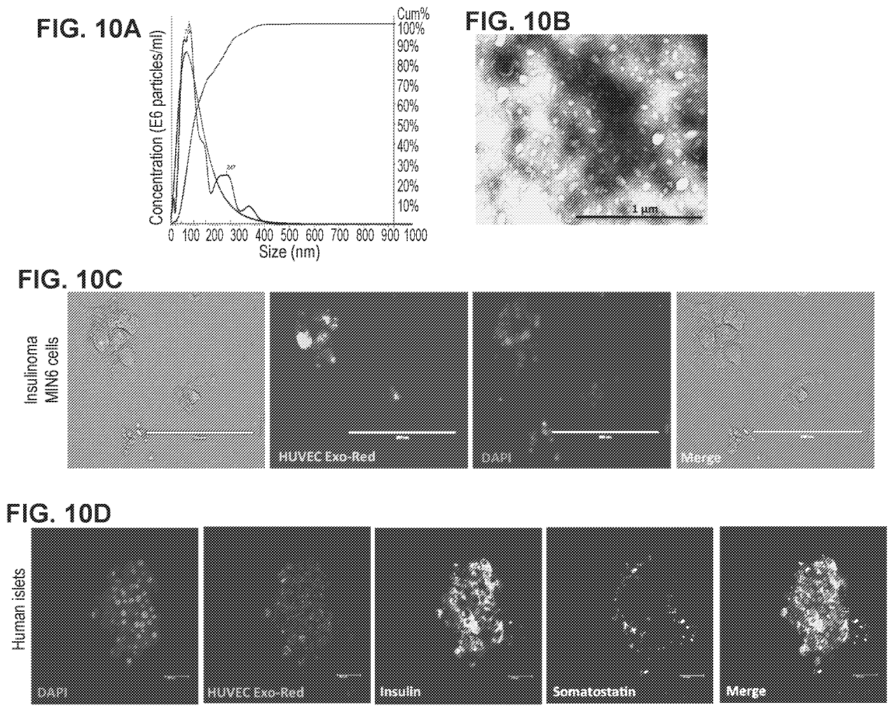

To determine if HUVEC-derived exosomes had an effect on islet survival and function, HUVEC cells were cultured and HUVEC-derived exosomes were prepared from conditioned media by ultracentrifugation. HUVEC were analyzed by phase-contrast microscopy and showed a typical cobblestone-pattern morphology with large nuclei. Exosomes were characterized by transmission electron microscopy (TEM) and Nanoparticle tracking analysis (NTA). NTA (FIG. 10A) confirmed the observed particle size and size distribution, the modal sizes of the HUVEC exosomes were estimated to be 96 nm. TEM analysis of exosomes showed vesicles at sizes <100 nm with the characteristic cup-shaped morphology (FIG. 10B) consistent with NTA data. Importantly, co-incubation of exosomes with cargo RNA labeled with Exo-Red (red stain) for 2 to 4 hours with MIN6 cells or human islets demonstrated uptake of exosomal RNA by the target cells (FIGS. 10C and 1D)

Islet Morphology

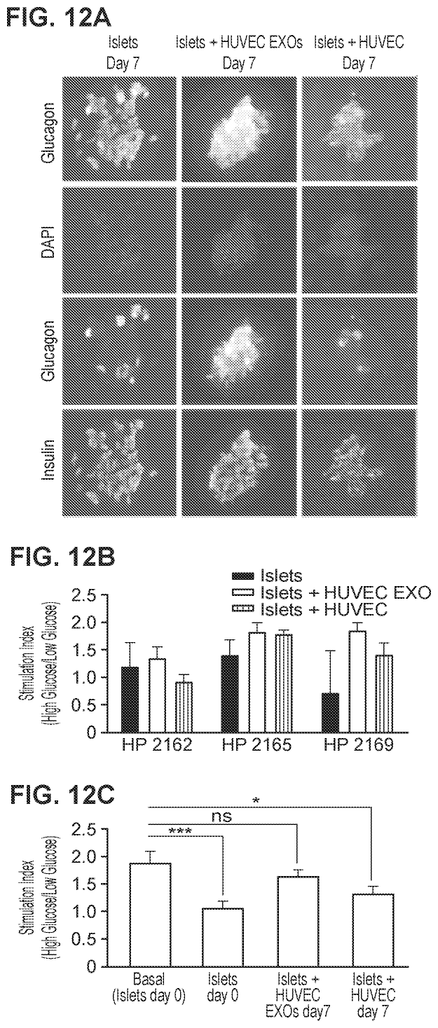

It is known that the preservation of human islet morphology is an important parameter for a prediction of islet function^{19,20}. Disrupted islet morphology and loss of islet function has been well established with progressing diabetes as well as with failure in islet transplantation outcome²¹. Human Islet morphology can be observed by phase-contrast microscopy as a small, discrete golden-brown cluster-like structure, approximately 50-250 μm in diameter, of endocrine cells surrounded by a capsule consisting of a single layer of fibroblasts after isolation. However, islets show morphological changes during 1 week in culture. When cultured alone islets gradually lose the three-dimensional cluster appearance and reduce to two-dimensional structure, which is associated with the loss of the membrane that surround them (FIG. 11). The number of cluster-like structures was reduced in non-treated islets and more irregular, flat and fragmented islets were found (FIG. 11A, a; FIG. 12A). Nevertheless, in islets treated with HUVEC or HUVEC-derived exosomes, cluster-like morphology was maintained and less migratory cells were found outside the islets (FIG. 11A, b and c, FIG. 12A).

Pancreatic Islet Functionality

Islet function was evaluated by glucose stimulated insulin secretion by comparing the percentages of cellular insulin released in low glucose (2.2 mM) versus high glucose (16.6 mM) media (FIG. 12B-C). Islets cultured for 1 week in standard conditions showed a significant decrease in the stimulation index (p value <0.0001) compared with islets at day 0 of culture. There was no difference in the simulation index of the Islets co-cultured with HUVEC derived-exosomes and a slight improvement in the stimulation index of islets co-cultured with HUVEC (p value <0.05) compared with the standard culture group at day 0. The greatest glucose-dependent insulin secretion occurred in islets treated with HUVEC-derived exosomes compared to non-treated islets after 7 days in culture (p<0.005; FIG. 12C).

Human Apoptosis Protein Array from Endothelial Cells and Endothelial Cell-Derived Exosomes.

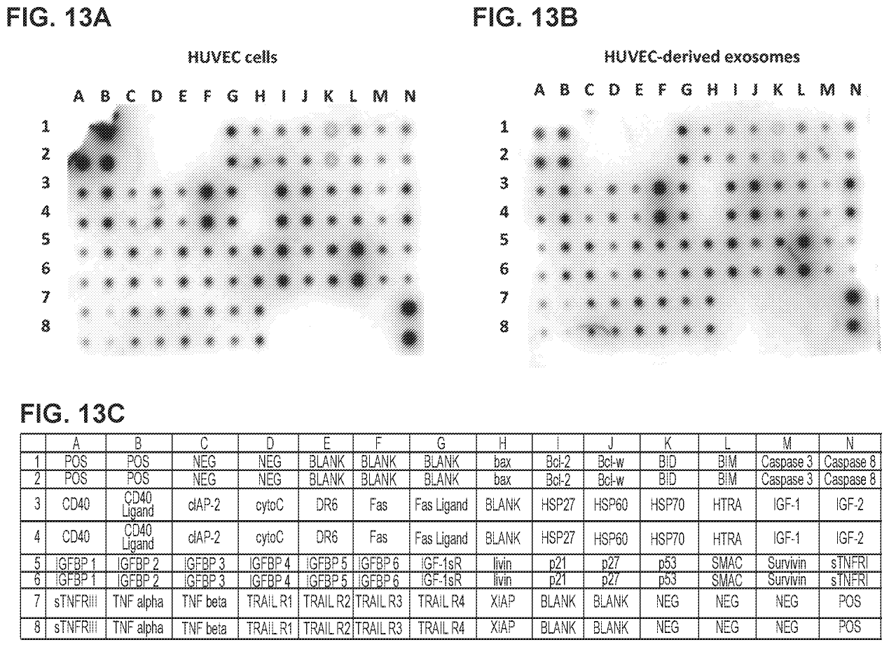

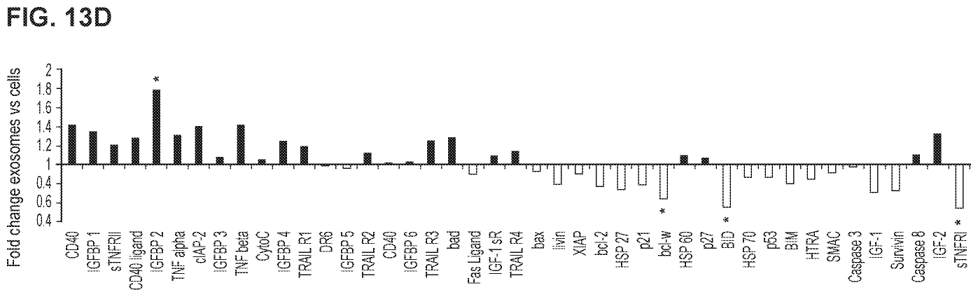

Having established that HUVEC or HUVEC-derived exosomes can enhance islet function in vitro, we further examined whether cells or exosomes could transfer anti-apoptotic proteins that could enhance islet function and provide protection to human islets. A human apoptosis-related protein array was performed to semi-quantitatively detect the presence of apoptosis-related proteins in cells and exosomes. The data from representative arrays are shown in FIG. 13. Quantification of protein signals by densitometry revealed Insulin-Like Growth Factor Binding Protein 2

IGFBP2 increased 1.8-fold in exosomes relative to levels in cells. In contrast, bcl-w (BCL2-like 2) decreased 0.64-fold, BID (BH3 interacting domain death agonist) decreased 0.55-fold and sTNFRI (soluble tumor necrosis factor receptor 1) decreased 0.53-fold (FIG. 13D).

Discussion

Although Islet transplantation is a promising therapeutic option for patients with diabetes, there is an urgent need to improve islet cell survival and viability during the in vitro cultured period pre-transplant. The aim of this study was to investigate the capability of endothelial cells to preserve and enhance the function of cultured human pancreatic islets. Here we demonstrated that human endothelial cell-derived exosomes facilitate the interaction between endothelial cells and islets. Moreover we demonstrated that exosomes released by human endothelial cells mediate the transfer and internalization of the RNA cargo carried by the exosomes into human islet cells. Furthermore, glucose stimulated insulin secretion revealed a 0.5 times higher stimulation index (p value <0,005) in the group of islets cultured for one week with endothelial-derived exosomes compared with those cultured under standard conditions. Taken together, these results demonstrate the ability of endothelial-derived exosomes to be internalized into human islets to preserve islet function.

The maintenance of islets in long-term culture is required to perform quality controls and shipment of islets for transplantation and has been shown to be associated with islet fragmentation, islet mass loss, and decrease in cell viabiity^{4,22}. An improvement of the quality of cultured islets pre-transplant should improve the success of islet transplantation and clinical outcome. Our results suggest that endothelial cells and, in particular, endothelial cell exosomes not only improve islet survival and function in culture, but also could contribute to normal pancreatic beta cell function in vivo_{7,13,23-26}. Endothelial cells have positive effects in the islet microenvironment through paracrine effects secreting molecules such as hepatocyte growth factors (HGF), thrombospondin-1(TSP-1), laminins, collagens and others which affect the function of islets. A similar positive effect on islets also could be mediated by endothelial cell exosomes in vivo.

In recent years, exosomes have emerged as potential candidates for mediating cell-cell communication in various physiological or pathophysiological conditions through transfer of proteins, mRNAs, and miRNAs^{17,28,29}. In this study, it was observed that endothelial cells release exosomes that can be isolated by ultracentrifugation displaying typical characteristics of exosomes, including a size between 30-100 nm and the cup-shape morphology. Moreover, it was demonstrated that endothelial-derived exosomes transfer their labeled RNA cargo to islets, thus mediating the transfer of biological information between both cell types. Moreover, the morphology of islets in the co-culture groups revealed maintenance of integrity, whereas there were more disrupted islets with irregular structures in the standard culture conditions. In addition to maintaining morphology, exosomes released by endothelial cells also demonstrated the ability to increase glucose-stimulated insulin secretion by beta pancreatic islet cells after 1 week in culture compared with islets cultured in standard conditions. Analysis of the differentially expressed proteins from the human apoptosis array of HUVEC and HUVEC-derived exosomes showed an increased level in exosomes of proteins involved in the positive regulation of cellular metabolic process, such as IGFBP2 related with antidiabetic effects³⁰, and new vessel formation and improvement of ischemic conditions³¹. In contrast, down-regulated proteins were correlated with extrinsic apoptotic signaling pathways, such as bcl-w pro-survival protein implicated in mitochondrial function and cell death³², BID implicated in FAS-dependent human islet cell death³³ and STNFRI related to cell death pathways³⁴, were decreased. Our findings from antibody array showed that exosomes released by endothelial cells release and/or downregulate expression of anti-apoptotic proteins at levels that may account for the higher therapeutic effect of HUVEC-derived exosomes.

Collectively, the findings described herein highlight a novel mode of paracrine communication between endothelial cells and human islets and identify a novel function for exosomes in the improvement of islet function in the treatment of diabetes mellitus. The paracrine effects of exosomes suggest that they could be used as a novel cell free-therapy to improve islet function pre-transplantation.

### Example 5

This example demonstrates the materials and methods utilized for the study described in Example 4.

Ethics Statement

This study was conducted according to the principles expressed in the Declaration of Helsinki. Pancreata from deceased heart-beating organ donors were obtained from various Organ Procurement Organizations in accordance with federal guidelines. Organs were processed for the isolation of islets at the cGMP facility of the Diabetes Research Institute, University of Miami, under Human Subjects Research Office of the University of Miami issued waiver that states that islet cells isolation from deceased donors does not constitute humane subject research.

Islet Isolation and Culturing

Islets isolated from three independent donors were used for the study. Table 2 summarizes the characteristics of the islet donors. Islets from human pancreas were isolated at the cGMP Human Islet Cell Processing Facility of the Diabetes Research Institute at the Miller School of Medicine, University of Miami. Human Islets were cultured overnight in supplemented CMRL-1066 medium (Gibco) at 22° C. and 5% CO₂.

TABLE 2

| Donor | HP2162 | HP2165 | HP2169 |
|---|---|---|---|
| Diabetic/Non- | NO | NO | NO |
| diabetic | | | |
| Age (years) | 27 | 41 | 37 |
| Gender | Female | Male | Female |
| ABO/Rh | A− | O+ | A |
| Race | C | C | C |
| Weight (kg) | 61 | 83.6 | 77.7 |
| Height (m) | 1.57 | 1.63 | 1.57 |
| BMI | 23.9 | 31.4 | 31.3 |
| Cold | 16 h 50 min | 12 h | 24 h 10 min |
| Ischemia | | | |
| Time | | | |
| Purity (%) | 80 | 87.5 | 95 |
| Viability (%) | 95.1 | 92.1 | 93 |

Cell Culture

Human umbilical vein endothelial cells (HUVEC) (ATCC). HUVEC were cultured in 200PRF Medium supplemented with LSGS (reference S-003-10, Lot 1541725 Gibco) and 1% antibiotics and were used during passages 4-5.

Pancreatic beta MIN6 cells (kindly provided by Dr. Buchwald, Diabetes Research Institute, Miami, Fla.) were cultured in Dulbecco's Modified Eagle's Medium (DMEM) containing 25 mM glucose, 10% (v/v) fetal bovine serum (FBS), penicillin (100 U/ml), streptomycin (100 μg/ml) (Sigma), 50 μM 2-mercaptoethanol (Gibco) at 37° C. in an atmosphere of 95% air and 5% C02.

IncuCyte Real-Time Imaging Assay

Human islets co-cultures plate, was inserted into the IncuCyte (ESSEN BioScience Inc) for real-time imaging, with sixteen fields imaged per well under 10× magnification every day for a total of one week. Data were analyzed using the IncuCyte Confluence version 1.5 software. All IncuCyte experiments were performed in triplicate.

Exosome-Depleted Media Preparation and Exosome Isolation

Medium supplemented with all nutrient was centrifuged 16-18 hours at 110,000×g, at 4° C., then the supernatant was filtered through a 0.22-μm filter into a sterile bottle. The media was stored up to 4 weeks at 4° C. HUVEC cells were grown under the usual conditions until they reached 80% confluence. The culture medium was removed and replaced with a similar volume of exosome-production medium. Cells were returned into the incubator for 48 hours.

Exosomes were isolated by ultra-centrifugation as described previously³⁵. The medium was centrifuged 10 min at 300×g for 10 min at 2000×g and 30 min at 10,000×g at 4° C. Then the supernatant was centrifuged 70 min at 100,000×g, at 4° C. The pellet in each tube was resuspended in 1 ml Phosphate Saline Buffer (PBS) and finally samples were centrifuged 1 hr at 100,000×g at 4° C. and the exosome pellet was resuspended in 200 μl of PBS.

Transmission Electron Microscopy

Purified exosomes were fixed with 1% glutaraldehyde in PBS. After rinsing, a 20 mL drop of the suspension was loaded onto a formvar/carbon-coated grid, negatively stained with 3% (w/v) aqueous phosphotungstic acid for 1 min, and observed by transmission electron microscopy (TEM) in a Tecnai Spirit G-2 apparatus; (FEI, Eindhoven, The Netherlands).

Nanoparticle Tracking Analysis (NTA)

Exosome preparations were diluted in sterile PBS and analyzed by Nanosight NS300 and Nanosight NTA 2.3 Analytical Software (Malvern Instruments company, Nanosight, and Malvern, United Kingdom) to obtain particle number and size distribution. At least three analyses were performed for each individual sample.

Co-Cultures

HUVEC cells were enzymatically detached from culture plates, counted, and added (0.1×10⁶ cells) to a 24 well culture dish (Corning) with approximately 300 human islets in a total volume of 1 mL. The same methodology was follow for addition of exosomes (1,29×10¹2 particles/ml). Controls included islets and HUVEC cultured alone.

Exosome Labeling and Live Imaging of Exosome Uptake

Exosome RNA cargo was labeled with Exo-Red (System Biosciences), according to manufacturer's protocol. Briefly, exosome pellets were resuspended in 100 μl of PBS, mixed with 10 μl of Exo-Red stain solution, an incubated for ten minutes at 37° C., then 100 of ExoQuick-TC reagent were added to stop the reaction. Labeled exosomes were incubated 30 minutes at 4° C. Finally exosomes were centrifuged 3 minutes at 14,000 rpm. Human Islets and MIN6 cells were grown in glass bottom dishes, and labeled exosomes were added and incubated for 1 hr. Cells were immediately imaged with a Leica SP5 confocal microscope.

Immunofluorescence Staining

Cells were fixed by adding 0.5 ml/well of 4% paraformaldehyde for 20 minutes at room temperature. Cells were washed in PBS and permeabilized in 0.5% Triton X-100 (Sigma) in 0.1 M PBS for 20 minutes. Cells were rinsed again in PBS. Blocking was done with 10% normal donkey serum and 0.1% Triton X-100 diluted in 0.1M PBS. Incubation with primary antibody mix was performed at 4° C. overnight, followed by incubation with the secondary antibodies at room temperature for 1 hour. The following primary antibodies were used: anti-Insulin (A0564 Dako), and anti-glucagon (A0565 Dako) at 1:250, somatostatin (DAKO A0566). Appropriate secondary antibodies coupled to Alexa 568 (A11075 Invitrogen), Alexa 488 (A11008 Invitrogen) and Alexa 647 (A31573 Invitrogen) were used at a dilution of 1:500. Both primary and secondary antibodies were diluted in blocking buffer. Nuclei were stained with 4′-6-diamidino-2-phenylindole (DAPI).

Glucose Stimulated Insulin Secretion Assay (GSIS)

A static incubation assay was used to determine glucose responsiveness in islet controls and in islet co-cultured with HUVEC or HUVEC-derived exosomes. Islets were placed in quadruplicates. Islets, islet-HUVEC co-cultures and islet-HUVEC derived exosomes co-cultures were equilibrated by incubating the cells in low-glucose (2.2 mM)-modified Krebs buffer containing 0.1% wt/vol BSA, 26 mM sodium bicarbonate and 25 mM HEPES buffer. Sequential 1-hour incubations were then conducted in low (2.2 mM), high (16.6 mM), and low (2.2 mM) buffers. Samples of medium were collected at the end of each hour following the pre-incubation, the islets were sedimented and the condition medium collected from each well was frozen at −80° C. until the insulin concentration was quantified using the Mercodia Human Insulin ELISA (Winston-Salem, N.C.).

Human Apoptosis-Related Protein Array

HUVEC cells and HUVEC-derived exosomes were analyzed using a human apoptosis antibody array (RayBiotech) according to the manufacturer's instructions. Briefly, the membranes were first blocked for 30 minutes at room temperature and then 1 ml (2,000 μg/mL) of proteins from either cells or exosomes was added and incubated overnight. The membranes were washed and 1 ml of primary biotin-conjugated antibody was added and incubated at room temperature for 2 h. Then, the membranes were washed, incubated with horseradish peroxidase-conjugated streptavidin for 2 hours at room temperature and washed again. Last, the membranes were incubated for 1 minute with the detection buffer after which the signal was detected with FluorChem E FE0506 imaging system (Alpha Innotech Corp.). The chemiluminescent signal was quantified using Image J. The relative expression levels of the proteins were obtained by comparing the signal intensities. However, the apoptosis antibody array is a semi quantitative method, direct conclusions about the concentrations cannot be drawn. The signal intensities of the positive controls on the array membranes were set as 100 and were used to normalize the results from the different membranes being compared. The relative expression levels of proteins in the samples were compared between HUVEC cells and HUVEC-derived exosomes. Any 1.5 fold-increases or 0.65-fold decreases in signal intensity were considered significant as per manufacturer's instructions.

Statistics

All experimental procedures are expressed as mean±SD. Statistical analysis was performed unpaired Student's ttest with Welch's correction applied when variances were significantly different. Human Apoptosis-related protein array statistical analysis was performed detecting the density of individual cytokines in all subjects in duplicate.

### Example 6

This example demonstrates an exemplary method of preparing islet cells prior to implanting into a subject in need thereof.

Human islet cells were co-cultured with (i) mesenchymal stem cells (MSC), (ii) exosomes derived from MSC, or (iii) MSC-conditioned medium were cultured, or a control, for 48 hours. About 500 IEQ pancreatic islet cells were cultured with about 500 MSC or about 10 ug/ml exosomes.

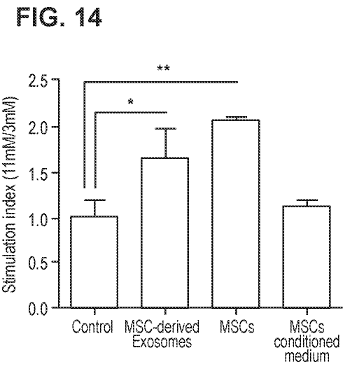

The effects of the co-culturing was evaluated by assaying the islets for glucose-stimulated insulin secretion (a 3 mM-to-11 mM glucose stimulation). FIG. 14 is a graph of the stimulation indices for each culture. As shown in FIG. 14, glucose-stimulated insulin secretion increased, relative to control, when the islets were co-cultured with MSCs or exosomes derived from MSCs. The glucose-stimulated insulin secretion of islets co-cultured with MSC-conditioned medium was similar to that of the control.

The effects of the co-culturing was also evaluated by assaying islet protein expression and comparing the protein expression profiles among the 4 groups of cell cultures. Co-culturing islets with MSC or exosomes derived from MSC caused an increased expression of inflammatory and immunomodulatory molecules. For example, the expression of Interleukin-6 (IL-6) and Interleukin-8 (IL-8), as well as CXCL3 (also known as MIP2b, GROg or GRO3), increased by islets co-cultured with MSC, relative to control islet cultures.

This example demonstrates that co-culturing islets with MSCs or exosomes derived from MSCs increases the functionality of islets.

### Example 7

This example demonstrates an exemplary method of preparing islet cells prior to implanting into a subject in need thereof.

AD-MSC were stimulated with various agents and then segregated into different phenotypes based on the molecular profiles of the exosomes secreted by the stimulated AD-MSC. In one instance, the AD-MSC were stimulated with 10 ng TNFα or 10 ng IFNγ in culture for about 48 hours. In another instance, AD-MSC were stimulated with lower amounts of these molecules (10 μg TNFα or 10 μg IFNγ) in culture for about 48 hours. Other stimulants included polyinosinic:polycytidylic acid (poly I:C) (50 μg/ml) and lipopolysaccharide (LPS) (1 μg/ml). An unstimulated control group of AD-MSC was maintained as well.

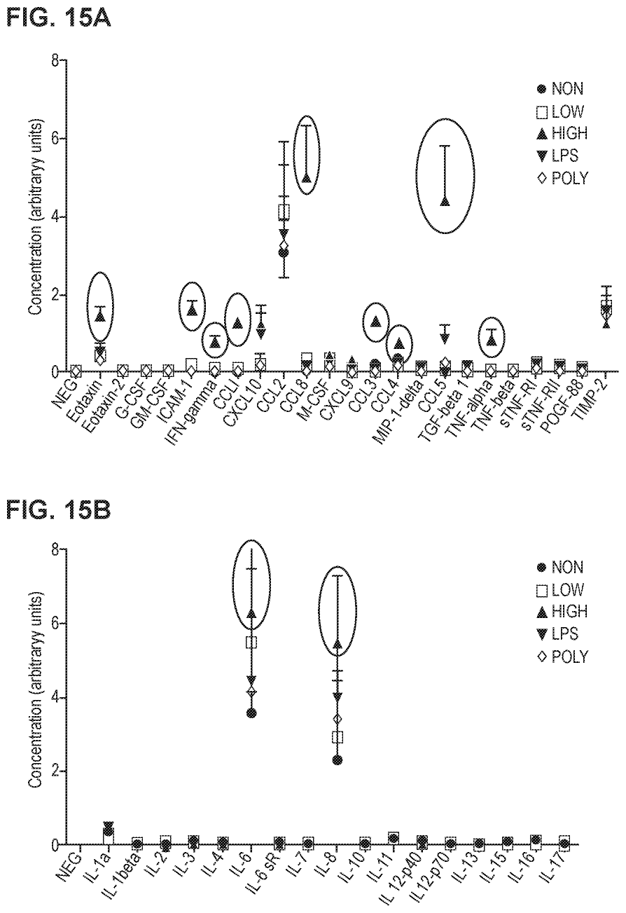

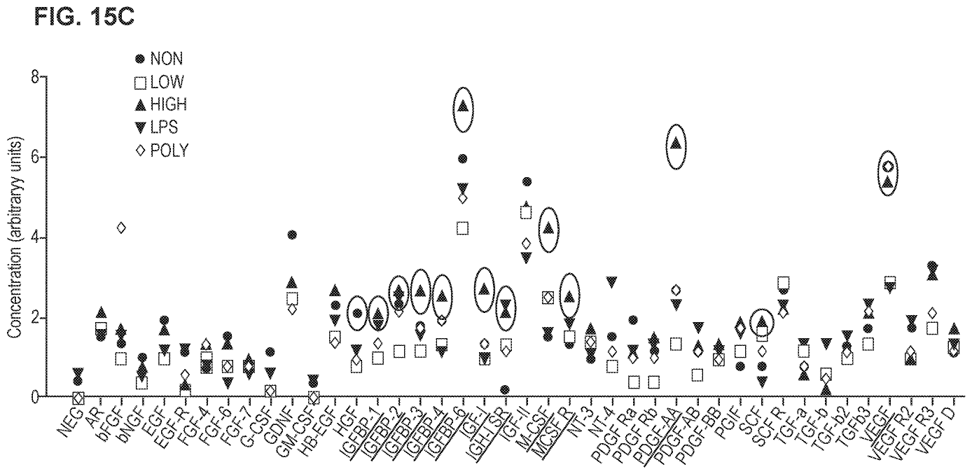

Cells were analyzed by flow cytometry or other techniques for molecular expression profiles. For example, a protein expression profile for each stimulated AD-MSC group and the unstimulated control group was determined. The results of such assays demonstrated that the various stimulants caused the AD-MSC to have different molecule expression profiles. Also, the expression profiles of the exosomes derived from the AD-MSC differed according to the stimulant, or lack of stimulus. For example, expression of the cell surface marker, CD146, was significantly increased when stimulated with the higher amount of TNFα or IFNγ, relative to the lower amount (10 μg) of the same cytokines. Also, when stimulated with the higher amount of TNFα or IFNγ (10 ng), the expression of particular growth factors (e.g., IGFB-1, IGFB-2, IGFB-3, IGFB-4, IGFB-6, IGF-1, IGF-1 SR, IGF-II, M-CSF, MCSF R, PDGF-AA, and VEGF) and particular inflammation regulators (e.g., IL-6, IL-8, Eotaxin, ICAM-1, IFNγ, CCL, MCP-2, MIP-1α, RANTES, and TNFα) was increased. See FIG. 15.

This example demonstrates a method of preparing MSC for co-implantation with islets.

### REFERENCES

The following references are cited in the Background and Examples 1 and 2.
1. Piemonti and Pileggi, CellR4 2013; 1:8-22
2. Pileggi et al., Twenty years of clinical islet transplantation at the Diabetes Research Institute—University of Miami. Clin Transpl 2004; 177-204pmid:167041500penUrlMedline
3. Alejandro R, et al. Natural history of intrahepatic canine islet cell autografts. J Clin Invest 1986; 78:1339-1348
4. Bennet W, et al., Isolated human islets trigger an instant blood mediated inflammatory reaction: implications for intraportal islet transplantation as a treatment for patients with type 1 diabetes. Ups J Med Sci 2000; 105:125-133
5. Moberg et al. Production of tissue factor by pancreatic islet cells as a trigger of detrimental thrombotic reactions in clinical islet transplantation. Lancet 2002; 360:2039-2045
6. Johansson et al. Tissue factor produced by the endocrine cells of the islets of Langerhans is associated with a negative outcome of clinical islet transplantation. Diabetes 2005; 54:1755-1762
7. Berman et al. Interference with tissue factor prolongs intrahepatic islet allograft survival in a nonhuman primate marginal mass model. Transplantation 2007; 84:308-315
8. Markmann et al. Magnetic resonance-defined periportal steatosis following intraportal islet transplantation: a functional footprint of islet graft survival?Diabetes 2003; 52:1591-1594
9. Bhargava et al. Prevalence of hepatic steatosis after islet transplantation and its relation to graft function. Diabetes 2004; 53:1311-1317
10. Eckhard M et al. Disseminated periportal fatty degeneration after allogeneic intraportal islet transplantation in a patient with type 1 diabetes mellitus: a case report. Transplant Proc 2004; 36:1111-1116
11. Maffi et al., Minimal focal steatosis of liver after islet transplantation in humans: a long-term study. Cell Transplant 2005; 14:727-733
12. Leitio et al. Liver fat accumulation after islet transplantation and graft survival. Cell Transplant 2014; 23:1221-1227
13. Jackson et al. Long-term follow-up of hepatic ultrasound findings in subjects with magnetic resonance imaging defined hepatic steatosis following clinical islet transplantation: a case-control study. Islets 2013; 5:16-21
14. Fotino et al., Re-engineering islet cell transplantation. Pharmacol Res 2015; 98:76-85
15. Pileggi and Ricordi, A new home for pancreatic islet transplants: the bone marrow. Diabetes 2013; 62:3333-3335
16. Philosophe et al., Superiority of portal venous drainage over systemic venous drainage in pancreas transplantation: a retrospective study. Ann Surg 2001; 234:689-696
17. Platell et al., The omentum. World J Gastroenterol 2000; 6:169-176
18. Chaffanjon et al., Omental anatomy of non-human primates. Surg Radiol Anat 2005; 27:287-291pmid:162374870penUrlCrossRefMedline
19. Ackermann et al., Microcirculation of the rat omentum studied by means of corrosion casts. Acta Anat (Basel) 1991; 140:146-149pmid:18670560penUrlCrossRefMedline
20. Guan et al., Liver-omental pouch and intrahepatic islet transplants produce portal insulin delivery and prevent hyperinsulinemia in rats. Transplant Proc 1995; 27:3236
21. Guan et al., Glucose turnover and insulin sensitivity in rats with pancreatic islet transplants. Diabetes 1998; 47:1020-1026
22. Berman et al., Long-term survival of nonhuman primate islets implanted in an omental pouch on a biodegradable scaffold. Am J Transplant 2009; 9:91-104
23. Kim et al., Comparison of four pancreatic islet implantation sites. J Korean Med Sci 2010; 25:203-210pmid:20119571OpenUrlCrossRefMedline
24. Perez et al., The anterior chamber of the eye as a clinical transplantation site for the treatment of diabetes: a study in a baboon model of diabetes. Diabetologia 2011; 54:1121-1126
25. Pileggi et al., Reversal of diabetes by pancreatic islet transplantation into a subcutaneous, neovascularized device. Transplantation 2006; 81:1318-1324
26. Ichii et al., Rescue purification maximizes the use of human islet preparations for transplantation. Am J Transplant 2005; 5:21-30
27. Hogan et al., Beneficial effects of ischemic preconditioning on pancreas cold preservation. Cell Transplant 2012; 21:1349-1360
28. Ricordi C, et al., Automated method for isolation of human pancreatic islets. Diabetes 1988; 37:413-420
29. Ricordi, Islet transplantation: a brave new world. Diabetes 2003; 52:1595-1603
30. Purified human pancreatic islets (PHPI) master production batch record—a standard operating procedure of the NIH Clinical Islet Transplantation Consortium. CellR4 2014; 2:e891
31. Pileggi A, et al., Impact of pancreatic cold preservation on rat islet recovery and function. Transplantation 2009; 87:1442-1450
32. Posselt et al., Islet transplantation in type 1 diabetic patients using calcineurin inhibitor-free immunosuppressive protocols based on T-cell adhesion or costimulation blockade. Transplantation 2010; 90:1595-1601
33. Vergani et al., A novel clinically relevant strategy to abrogate autoimmunity and regulate alloimmunity in NOD mice. Diabetes 2010; 59:2253-2264
34. Badell et al., CTLA4Ig prevents alloantibody formation following nonhuman primate islet transplantation using the CD40-specific antibody 3A8. Am J Transplant 2012; 12:1918-1923
35. Safley et al., Encapsulated piscine (tilapia) islets for diabetes therapy: studies in diabetic NOD and NOD-SCID mice. Xenotransplantation 2014; 21:127-139
36. Brady et al., Local immumosuppression modulates islet allograft rejection into a biohybrid device. Am J Transplant 2010; 10:76-77
37. Faradji et al., C-peptide and glucose values in the peritransplant period after intraportal islet infusions in type 1 diabetes. Transplant Proc 2005; 37:3433-3434
38. Berney et al., Detection of insulin mRNA in the peripheral blood after human islet transplantion predicts deterioration of metabolic control. Am J Transplant 2006; 6:1704-1711
39. Yasunami et al., A new site for islet transplantation—a peritoneal-omental pouch. Transplantation 1983; 36:181-182
40. Simeonovic et al., A comparative study of transplant sites for endocrine tissue transplantation in the pig. Aust J Exp Biol Med Sci 1986; 64:37-41
41. Ao et al., Development of an omental pouch site for islet transplantation. Transplant Proc 1992; 24:2789
42. Ao et al., Survival and function of purified islets in the omental pouch site of outbred dogs. Transplantation 1993; 56:524-529
43. Jacobs-Tulleneers-Thevissen et al., Human islet cell implants in a nude rat model of diabetes survive better in omentum than in liver with a positive influence of beta cell number and purity. Diabetologia 2010; 53:1690-1699
44. Bartholomeus et al., Omentum is better site than kidney capsule for growth, differentiation, and vascularization of immature porcine β-cell implants in immunodeficient rats. Transplantation 2013; 96:1026-1033
45. Pedraza et al., Macroporous three-dimensional PDMS scaffolds for extrahepatic islet transplantation. Cell Transplant 2013; 22:1123-1135
46. Ham et al., Thrombin use in surgery: an evidence-based review of its clinical use. J Blood Med 2010; 1:135-142
47. Hefty et al., Omental roll-up: a technique for islet engraftment in a large animal model. J Surg Res 2010; 161:134-138pmid:193946490penUrlCrossRefMedline
48. Cray et al., Acute phase response in animals: a review. Comp Med 2009; 59:517-526
49. Procaccini et al., Neuro-endocrine networks controlling immune system in health and disease. Front Immunol 2014; 5:143
50. Ozmen et al., Inhibition of thrombin abrogates the instant blood-mediated inflammatory reaction triggered by isolated human islets: possible application of the thrombin inhibitor melagatran in clinical islet transplantation. Diabetes 2002; 51:1779-1784pmid:120319650penUrlAbstract/FREE Full Text
51. Ichii et al., Characterization of pancreatic ductal cells in human islet preparations. Lab Invest 2008; 88:1167-1177
52. Ricordi et al., Long-term in vivo function of human mantled islets obtained by incomplete pancreatic dissociation and purification. Transplant Proc 1995; 27:3382
53. Street et al., Islet graft assessment in the Edmonton Protocol: implications for predicting long-term clinical outcome. Diabetes 2004; 53:3107-3114
54. Chinnakotla et al., Total pancreatectomy and islet autotransplantation in children for chronic pancreatitis: indication, surgical techniques, postoperative management, and long-term outcomes. Ann Surg 2014; 260:56-64
55. Kakabadze et al. Correction of diabetes mellitus by transplanting minimal mass of syngeneic islets into vascularized small intestinal segment. Am J Transplant 2013; 13:2550-2557

The following references are cited in Example 3.
1. Hering B J, Clarke W R, Bridges N D, et al. Phase 3 Trial of Transplantation of Human Islets in Type 1 Diabetes Complicated by Severe Hypoglycemia. Diabetes Care 2016; 39:1230-40.
2. Cantarelli E, Piemonti L. Alternative transplantation sites for pancreatic islet grafts. Curr Diab Rep 2011; 11:364-74.
3. Berman D M, O'Neil J J, Coffey L C, et al. Long-term survival of nonhuman primate islets implanted in an omental pouch on a biodegradable scaffold. Am J Transplant 2009; 9:91-104.
4. Berman D M, Molano R D, Fotino C, et al. Bioengineering the Endocrine Pancreas: Intraomental Islet Transplantation Within a Biologic Resorbable Scaffold. Diabetes 2016; 65:1350-61.
5. Forbes S, Oram R A, Smith A, et al. Validation of the BETA-2 Score: An Improved Tool to Estimate Beta Cell Function After Clinical Islet Transplantation Using a Single Fasting Blood Sample. Am J Transplant 2016; 16:2704-13.

The following references are cited in the Supplementary Information of Example 3:
1. Clarke W L, Cox D J, Gonder-Frederick L A, Julian D, Schlundt D, Polonsky W. Reduced awareness of hypoglycemia in adults with IDDM. A prospective study of hypoglycemic frequency and associated symptoms. Diabetes Care 1995; 18:517-22.
2. Poggioli R, Enfield G, Messinger S, et al. Nutritional status and behavior in subjects with type 1 diabetes, before and after islet transplantation. Transplantation 2008; 85:501-6.
3. Wallace T M, Levy J C, Matthews D R. Use and abuse of HOMA modeling. Diabetes Care 2004; 27:1487-95.
4. Ryan E A, Paty B W, Senior P A, Lakey J R, Bigam D, Shapiro A M. Beta-score: an assessment of beta-cell function after islet transplantation. Diabetes Care 2005; 28:3437.
5. Forbes S, Oram R A, Smith A, et al. Validation of the BETA-2 Score: An Improved Tool to Estimate Beta Cell Function After Clinical Islet Transplantation Using a Single Fasting Blood Sample. Am J Transplant 2016; 16:2704-13.

The following references are cited in Examples 4 and 5.
1. Robertson, R. P., Davis, C., Larsen, J., Stratta, R. & Sutherland, D. E. Pancreas and islet transplantation for patients with diabetes. Diabetes care 23, 112-116 (2000).
2. Piemonti Lorenzo, P. A. 25 Years of the Ricordi Automated Method for Islet Isolation. CellR4 2013; 1 (1): e128.
3. Tharavanij, T. et al. Improved long-term health-related quality of life after islet transplantation. Transplantation 86, 1161-1167, doi:10.1097/TP.0b013e31818a7f45 (2008).
4. Kin, T. et al. Risk factors for islet loss during culture prior to transplantation. Transplant international: official journal of the European Society for Organ Transplantation 21, 1029-1035, doi:10.1111/j.1432-2277.2008.00719.x (2008).
5. Ihm, S. H., Matsumoto, I., Zhang, H. J., Ansite, J. D. & Hering, B. J. Effect of short-term culture on functional and stress-related parameters in isolated human islets. Transplant international: official journal of the European Society for Organ Transplantation 22, 207216, doi:10.1111/j.1432-2277.2008.00769.x (2009).
6. Purified Human Pancreatic Islets (PHPI) Master Production Batch Record—A Standard Operating Procedure of the NIH Clinical Islet Transplantation Consortium. CellR4 2014; 2 (2): e891.
7. Song, H. J. et al. Improved islet survival and function with rat endothelial cells in vitro co-culture. Transplantation proceedings 41, 4302-4306, doi:10.1016/j.transproceed.2009.09.071 (2009).
8. Li, Y. et al. Combined strategy of endothelial cells coating, Sertoli cells coculture and infusion improves vascularization and rejection protection of islet graft. PloS one 8, e56696, doi:10.1371/journal.pone.0056696 (2013).
9. Sordi, V. et al. Mesenchymal cells appearing in pancreatic tissue culture are bone marrow-derived stem cells with the capacity to improve transplanted islet function. Stem cells 28, 140-151, doi:10.1002/stem.259 (2010).
10. Yamada, S. et al. Trophic effect of adipose tissue-derived stem cells on porcine islet cells. The Journal of surgical research 187, 667-672, doi:10.1016/j.jss.2013.10.031 (2014).
11. Bhang, S. H. et al. Mutual effect of subcutaneously transplanted human adipose-derived stem cells and pancreatic islets within fibrin gel. Biomaterials 34, 7247-7256, doi:10.1016/j.biomaterials.2013.06.018 (2013).
12. Johansson, U. et al. Formation of composite endothelial cell-mesenchymal stem cell islets: a novel approach to promote islet revascularization. Diabetes 57, 2393-2401, doi:10.2337/db07-0981 (2008).
13. Pan, X. et al. [Endothelial cells promote islet survival and function]. Zhong nan da xue xue bao. Yi xue ban=Journal of Central South University. Medical sciences 39, 129-135, doi:10.11817/j.issn.1672-7347.2014.02.004 (2014).
14. Narang, A. S., Sabek, O., Gaber, A. O. & Mahato, R. I. Co-expression of vascular endothelial growth factor and interleukin-1 receptor antagonist improves human islet survival and function. Pharmaceutical research 23, 1970-1982, doi:10.1007/s11095-006-9065-7 (2006).
15. Paget, M. B., Murray, H. E., Bailey, C. J., Flatt, P. R. & Downing, R. Rotational co-culture of clonal beta-cells with endothelial cells: effect of PPAR-gamma agonism in vitro on insulin and VEGF secretion. Diabetes, obesity & metabolism 13, 662-668, doi:10.1111/j.1463-1326.2011.01392.x (2011).
16. Cantaluppi, V. et al. Microvesicles derived from endothelial progenitor cells enhance neoangiogenesis of human pancreatic islets. Cell transplantation 21, 1305-1320, doi:10.3727/096368911X627534 (2012).
17. Figliolini, F. et al. Isolation, characterization and potential role in beta cell-endothelium cross-talk of extracellular vesicles released from human pancreatic islets. PloS one 9, e102521, doi:10.1371/journal.pone.0102521 (2014).
18. Thery, C., Zitvogel, L. & Amigorena, S. Exosomes: composition, biogenesis and function. Nature reviews. Immunology 2, 569-579, doi:10.1038/nri855 (2002).
19. Cabrera, O. et al. The unique cytoarchitecture of human pancreatic islets has implications for islet cell function. Proceedings of the National Academy of Sciences of the United States of America 103, 2334-2339, doi:10.1073/pnas.0510790103 (2006).
20. Morini, S. et al. Morphological changes of isolated rat pancreatic islets: a structural, ultrastructural and morphometric study. Journal of anatomy 209, 381-392, doi:10.1111/j.1469-7580.2006.00620.x (2006).
21. Rackham, C. L., Jones, P. M. & King, A. J. Maintenance of islet morphology is beneficial for transplantation outcome in diabetic mice. PloS one 8, e57844, doi:10.1371/journal.pone.0057844 (2013).
22. Olsson R, C. P. Better vascular engraftment and function in pancreatic islets transplanted without prior culture. Diabetologia 48 (2005).
23. Rackham, C. L. et al. Pre-culturing islets with mesenchymal stromal cells using a direct contact configuration is beneficial for transplantation outcome in diabetic mice. Cytotherapy 15, 449-459, doi:10.1016/j.jcyt.2012.11.008 (2013).
24. Kang, S. et al. Endothelial progenitor cell cotransplantation enhances islet engraftment by rapid revascularization. Diabetes 61, 866-876, doi:10.2337/db10-1492 (2012).
25. Johansson, M., Mattsson, G., Andersson, A., Jansson, L. & Carlsson, P. O. Islet endothelial cells and pancreatic beta-cell proliferation: studies in vitro and during pregnancy in adult rats. Endocrinology 147, 2315-2324, doi:10.1210/en.2005-0997 (2006).
26. Quaranta, P. et al. Co-transplantation of endothelial progenitor cells and pancreatic islets to induce long-lasting normoglycemia in streptozotocin-treated diabetic rats. PloS one 9, e94783, doi:10.1371/journal.pone.0094783 (2014).
27. Cao, Z. & Wang, X. The endocrine role between beta cells and intra-islet endothelial cells. Endocrine journal 61, 647-654 (2014).
28. Valadi, H. et al. Exosome-mediated transfer of mRNAs and microRNAs is a novel mechanism of genetic exchange between cells. Nature cell biology 9, 654-659, doi:10.1038/ncb1596 (2007).
29. Guay, C., Menoud, V., Rome, S. & Regazzi, R. Horizontal transfer of exosomal microRNAs transduce apoptotic signals between pancreatic beta-cells. Cell communication and signaling: CCS 13, 17, doi:10.1186/s12964-015-0097-7 (2015).
30. Hedbacker, K. et al. Antidiabetic Effects of IGFBP2, a Leptin-Regulated Gene (vol 6, pg 11, 2010). Cell Metab 11, 239-239, doi:10.1016/j.cmet.2010.02.010 (2010).
31. Feng, N. et al. Insulin-Like Growth Factor Binding Protein-2 Promotes Adhesion of Endothelial Progenitor Cells to Endothelial Cells via Integrin alpha5beta1. J Mol Neurosci 57, 426-434, doi:10.1007/s12031-015-0589-3 (2015).
32. Soria, B. & Gauthier, B. R. Dual Trade of Bcl-2 and Bcl-xL in islet physiology: balancing life and death with metabolism secretion coupling. Diabetes 62, 18-21, doi:10.2337/db12-1023 (2013).
33. Joglekar, M. V. et al. Human islet cells are killed by BID-independent mechanisms in response to FAS ligand. Apoptosis, doi:10.1007/s10495-016-1212-y (2016).
34. Stephens, L. A. et al. Tumor necrosis factor-alpha-activated cell death pathways in NIT-1 insulinoma cells and primary pancreatic beta cells. Endocrinology 140, 3219-3227, doi:10.1210/endo.140.7.6873 (1999).
35. Thery, C., Amigorena, S., Raposo, G. & Clayton, A. Isolation and characterization of exosomes from cell culture supernatants and biological fluids. Current protocols in cell biology/editorial board, Juan S. Bonifacino . . . [et al.]Chapter 3, Unit 3 22, doi:10.1002/0471143030.cb0322s30 (2006).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose alimitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed:

1. A method of implanting therapeutic cells in a subject, comprising:
a. combining the therapeutic cells with a source of a first member of a cell matrix pair to create a therapeutic cell mixture;
b. applying the therapeutic cell mixture to a surface of an organ in the subject;
c. applying a source of a second member of the cell matrix pair to the surface of the organ over the therapeutic cell mixture; and
d. applying a source of the first member of the cell matrix pair to the surface of the organ over the therapeutic cell mixture, thereby forming a therapeutic cell scaffold.

2. The method of claim 1, wherein the subject is a mammal.

3. The method of claim 2, wherein the mammal is a human.

4. The method of any one of claims 1 to 3, wherein the therapeutic cells produce and secrete a therapeutic agent.

5. The method of claim 4, wherein the therapeutic agent is a peptide, blood factor, hormone, growth factor, or cytokine.

6. The method of claim 5, wherein the subject has a deficiency in the therapeutic agent.

7. The method of any one of claims 1 to 6, wherein the therapeutic agent is insulin or an insulin analog.

8. The method of any one of the preceding claims, wherein the therapeutic cells are pancreatic islet cells, induced pluripotent stem cells (iPSC), embryonic stem cells, or cells genetically modified to produce and secrete insulin or an insulin analog.

9. The method of any one of the preceding claims, wherein the cell matrix pair is thrombin and fibrinogen.

10. The method of claim 9, wherein the source of the first member of the cell matrix pair is plasma.

11. The method of claim 10, wherein the plasma is autologous to the subject.

12. The method of any one of the preceding claims, further comprising collecting plasma from the subject.

13. The method of claim 12, further comprising administering to the subject one or more of an omega-3 fatty acid, an anti-inflammatory agent, an immunomodulatory molecule, a growth factor, or a combination thereof, prior to collecting plasma from the subject.

14. The method of any one of claims 1 to 11, wherein the plasma has been enriched for a platelet-rich plasma (PRP), anti-inflammatory factor, pro-angiogenic factor, growth factor, or a combination thereof.

15. The method of any one of claims 9 to 14, wherein the source of the second member of the cell matrix pair is recombinant human thrombin.

16. The method of claim 9, wherein the first member is thrombin and the source of the second member is plasma.

17. The method of any one of the preceding claims, wherein the organ is a mesothelial organ.

18. The method of claim 17, wherein the mesothelial organ is peritoneum.

19. The method of claim 17, wherein the mesothelial organ is a liver, pancreas, or an organ of the gastrointestinal tract.

20. The method of claim 17, wherein the mesothelial organ is an omentum.

21. The method of claim 20, comprising laparoscopically layering the therapeutic cell mixture along an axis of the omentum.

22. The method of claim 21, further comprising folding opposing sides of the omentum along the axis to envelope the therapeutic cell scaffold in the omentum.

23. The method of any one of claims 1 to 22, comprising combining the therapeutic cells with plasma to form a therapeutic cell mixture, applying the therapeutic cell mixture to the surface of the omentum of the subject, applying thrombin over the therapeutic cell mixture, and applying plasma over the therapeutic cell mixture and the thrombin.

24. The method of any one of the previous claims, wherein the subject suffers from a metabolic disease.

25. The method of claim 24, wherein the metabolic disease is diabetes or obesity.

26. The method of claim 25, wherein the diabetes is Type I diabetes.

27. The method of claim 26, wherein the Type I diabetes occurs with severe hypoglycemia.

28. The method of any one of claims 24 to 27, wherein the subject is treated for the metabolic disease with exogenous insulin therapy.

29. The method of claim 28, comprising ceasing insulin therapy after the therapeutic cells are implanted into the subject.

30. A method of preparing pancreatic islet cells for implantation into a subject, comprising culturing the pancreatic islet cells with endothelial-derived exosomes or endothelial cells.

31. The method of claim 30, wherein the endothelial cells are human umbilical vein endothelial cells (HUVEC) or the endothelial-derived exosomes are HUVEC-derived endosomes.

32. The method of claim 30 or 31, wherein the pancreatic islet cells are human pancreatic islet cells.

33. The method of any one of claims 30 to 32, wherein about 1000-5000 pancreatic islet cells are cultured with about 10⁴ to about 10⁶ endothelial cells or about 10¹⁰ to about 10¹⁵ exosomes, per ml.

34. The method of any one of claims 30 to 33, comprising culturing the pancreatic islet cells with endothelial-derived exosomes or endothelial cells for at least about 24 to about 48 hours.

35. The method of claim 34, comprising culturing the pancreatic islet cells with endothelial-derived exosomes or endothelial cells for at least about 3 to 6 days.

36. The method of claim 35, comprising culturing the pancreatic islet cells with endothelial-derived exosomes or endothelial cells for at least about 1 to about 2 weeks.

37. A method of preparing pancreatic islet cells for implantation into a subject, comprising culturing the pancreatic islet cells with contents of endothelial-derived exosomes prior to implanting the pancreatic islet cells into the subject.

38. A method of preparing pancreatic islet cells for implantation into a subject, comprising culturing the pancreatic islet cells with hepatocyte growth factor (HGF), thrombospondin-1 (TSP-1), a laminin, a collagen, insulin growth factor binding protein-1 (IGFBP-2), CD40, IGFBP-1, sTNFRII, CD40L, TNFα, cIAP-2, IGFBP-3, TNFβ, CytoC, IGFBP-4, TRAIL R1, TRAIL R2, TRAIL R3, bad, IGF-1 sR, TRAIL R4, HSP60, p27, Caspase 8, IGF-2, or a combination thereof, prior to implanting the pancreatic islet cells into the subject.

39. A method of preparing pancreatic islet cells for implantation into a subject, comprising culturing the pancreatic islet cells with mesenchymal stem cell (MSC)-derived exosomes or MSC.

40. The method of claim 39, wherein the MSC are adipose-derived MSC (AD-MSC).

41. The method of claim 39 or 40, wherein the MSC express CD146.

42. The method of any one of claims 39 to 41, wherein the MSC are stimulated with an inflammatory regulator.

43. The method of any one of claims 39 to 42, comprising culturing the pancreatic islet cells with MSC-derived exosomes or MSC for at least about 24 to about 48 hours.

44. The method of claim 43, comprising culturing the pancreatic islet cells with MSC-derived exosomes or MSC for at least about 3 to 6 days.

45. A method of preparing pancreatic islet cells for implantation into a subject, comprising culturing the pancreatic islet cells with contents of MSC-derived exosomes prior to implanting the pancreatic islet cells into the subject.

46. A method of preparing pancreatic islet cells for implantation into a subject, comprising culturing the pancreatic islet cells with IGFB-1, IGFB-2, IGFB-3, IGFB-4, IGFB-6, IGF-1, IGF-1 SR, IGF-II, M-CSF, MCSF R, PDGF-AA, VEGF, IL-6, IL-8, Eotaxin, ICAM-1, IFNγ, CCL1, MCP-2, MIP-1α, RANTES, TNFα, or a combination thereof, prior to implanting the pancreatic islet cells into the subject.

47. A method of implanting pancreatic islet cells in a subject, comprising:
a. preparing the pancreatic islet cells according to the method of any one of claims 30 to 46
b. combining pancreatic islet cells prepared in step (a) with a source of a first member of a cell matrix pair to create a therapeutic cell mixture;
c. applying the therapeutic cell mixture to a surface of an organ in the subject; and
d. applying a source of a second member of the cell matrix pair to the surface of the organ over the therapeutic cell mixture, thereby forming a therapeutic cell scaffold.

48. A method of implanting pancreatic islet cells in a subject, comprising:
a. combining pancreatic islet cells with a supporting cellular composition and a source of a first member of a cell matrix pair to create a therapeutic cell mixture;
b. applying the therapeutic cell mixture to a surface of an organ in the subject; and
c. applying a source of a second member of the cell matrix pair to the surface of the organ over the therapeutic cell mixture thereby forming a therapeutic cell scaffold.

49. The method of claim 48, wherein the supporting cellular composition comprises endothelial-derived exosomes or endothelial cells.

50. The method of claim 48 or 49, wherein the supporting cellular composition comprises immunomodulatory cells, stem cells, micronized fat cell clusters, bone marrow-derived mononuclear cells, cells of an adipose-derived stromal vascular fraction, endothelial progenitor cells, pericytes, hematopoietic progenitor cells, monocytes, leukocytes, fibroblastic reticular stromal cells, a fat tissue fraction comprising mesenchymal stem cells (MSC), or cell mixtures thereof.

51. The method of claim 50, wherein the immunomodulatory cells are Treg cells.

52. The method of claim 50, wherein the stem cells are MSC, bone marrow-derived stem cells, adipose tissue-derived stem cells, endothelial progenitor cells, or cell mixtures thereof.

53. The method of any one of claims 47 to 52, further comprising applying plasma to the surface of the organ over the therapeutic cell mixture.

54. The method of claim 53, comprising applying plasma to the surface of the organ after applying the thrombin.

55. The method of any one of the previous claims, wherein the therapeutic cells are microencapsulated, macroencapsulated, nanoencapsulated or conformally coated.

56. The method of claim 55, wherein the therapeutic cells are encapsulated with the supporting cell composition.

57. The method of claim 55, wherein the therapeutic cells are encapsulated separately from the supporting cell composition.

58. A method of reducing an exogenous insulin requirement (EIR) of a subject with diabetes, comprising implanting cells in the subject according to the method of any one of the preceding claims.

59. A method of restoring euglycemia in a subject in need thereof, comprising implanting cells in the subject according to the method of any one of the preceding claims.

60. A method of inducing insulin-independence or reducing insulin resistance in a subject, comprising implanting cells in the subject according to the method of any one of the preceding claims.

61. A method of inducing weight loss in a subject, comprising implanting cells in the subject according to the method of any one of the preceding claims.
